(19) United States
(12) Patent Application Publication
(10) Pub. No.: US 2025/0195798 A1
(43) Pub. Date: Jun. 19, 2025

(54) AN ELECTRONIC DEVICE FOR CHARACTERIZING AND/OR MONITORING AN INHALATION AND/OR AN EXHALATION WITH AN INHALER DEVICE, RELATED SYSTEM AND METHOD

(71) Applicant: SONOHALER ApS, Kongens Lyngby (DK)

(72) Inventors: Adam BOHR, Kongens Lyngby (DK); Troels TREBBIEN, Kongens Lyngby (DK); Benjamin EJLERTSEN, Kongens Lyngby (DK)

(73) Assignee: SONOHALER ApS, Kongens Lyngby (DK)

(21) Appl. No.: 18/859,729

(22) PCT Filed: Apr. 25, 2023
(86) PCT No.: PCT/EP2023/060787

§ 371 Date: Oct. 24, 2024

(30) Foreign Application Priority Data
Apr. 25, 2022 (EP) 22169711.3

(51) Int. Cl. A61M 15/00; G16H 20/13

(52) U.S. Cl. CPC A61M 15/0065; G16H 20/13; A61M 2205/3375; A61M 2230/42

(57) ABSTRACT

An electronic device for characterizing and/or monitoring an inhalation and/or an exhalation with an inhaler device is disclosed. The electronic device comprises a memory, an interface and a processor comprising predictor circuitry configured to operate according to a prediction model. The processor is configured to obtain inhalation data (such as inhalation and/or exhalation data), where the inhalation data is indicative of an audio signal representing an inhalation and/or an exhalation with the inhaler device. The processor is configured to determine, based on the inhalation data, using the predictor circuitry, a predicted inhalation parameter indicative of a prediction of an inhalation flow (such as inhalation flow and/or an exhalation flow) with the inhaler device. The processor is configured to determine, based on the predicted inhalation parameter, an inhalation representation.

The present disclosure pertains to the field of electronic devices, and in particular to electronic devices for characterizing and/or monitoring an inhalation and/or an exhalation with an inhaler device, related systems and related methods.

### BACKGROUND

Medication inhalers are diverse in their configuration and operation and many users have difficulties using them correctly and knowing whether they used a medication inhaler correctly. Currently, patients are left to themselves to learn and assess their inhalation technique with inhalers aside from a possible initial demonstration by their practitioner. Poor inhalation technique is likely to results in unsatisfactory treatment with any inhaler medication. Poor inhalation can be related to too low or too high inhalation flow, varying (such as fluctuating) inhalation flow, too short inhalation duration, poor coordination (such as poor coordination of an activation and/or release of a medication dose and inhalation), wrong usage of inhaler etc. Wrong usage of the inhaler may for example comprise inhaling from the wrong end of the inhaler, exhaling into the inhaler instead of inhaling, inhaling with too low inhalation flow in the beginning of a medication intake and then ending the medication intake with a too high inhalation flow, and/or having pauses in the inhalation during a medication intake. Adherence, such as lack of adherence, is another major problem for people with asthma and results in unnecessary hospitalization events and incurs great costs to the healthcare system and to society. Adherence can be improved but encouraging users to use their inhaler regularly and remind them to use the inhaler in case they forget to take it. Adherence is mainly related to control inhalers for asthma, typically anti-inflammatory medication that serves as a prophylactic treatment to prevent exacerbations and other undesirable events.

### SUMMARY

There is currently a lack of simple, accurate and convenient technologies for monitoring inhalation using inhalers (such as inhaler devices). There is also a lack in technologies that can directly interpret and/or determine the performance of an inhalation with an inhaler device. Also, there is no existing technology that can determine how and to what degree a medication dose was taken by the user.

Accordingly, there is a need for electronic devices for characterizing and/or monitoring an inhalation and/or an exhalation with an inhaler device, systems for characterizing and/or monitoring an inhalation and/or an exhalation with an inhaler device, and methods for characterizing and/or monitoring an inhalation and/or an exhalation with an inhaler device, which may mitigate, alleviate, or address the shortcomings existing and may provide improved characterization and/or monitoring of an inhalation and/or an exhalation with an inhaler device with improved feedback which is more intelligible for the user and with improved accuracy and precision.

An electronic device for characterizing and/or monitoring an inhalation and/or an exhalation with an inhaler device is disclosed. The electronic device comprises a memory, an interface and a processor comprising predictor circuitry configured to operate according to a prediction model. The processor is configured to obtain inhalation data (such as inhalation and/or exhalation data), where the inhalation data is indicative of an audio signal representing an inhalation and/or an exhalation with the inhaler device. The processor is configured to determine, based on the inhalation data, using the predictor circuitry, a predicted inhalation parameter indicative of a prediction of an inhalation flow (such as inhalation flow and/or an exhalation flow) with the inhaler device. The processor is configured to determine, based on the predicted inhalation parameter, an inhalation representation. Optionally, the processor is configured to output, via the interface, the inhalation representation.

Further, a system for characterizing and/or monitoring an inhalation and/or an exhalation with an inhaler device is disclosed. The system comprises the inhaler device and an electronic device as disclosed herein.

Further, a method, for characterizing and/or monitoring an inhalation and/or an exhalation with an inhaler device is disclosed. The method comprises obtaining inhalation data, where the inhalation data is indicative of an audio signal representing an inhalation and/or an exhalation with the inhaler device. The method comprises determining, based on the inhalation data, using the predictor circuitry, a predicted inhalation parameter indicative of a prediction of an inhalation flow with the inhaler device. The method comprises determining, based on the predicted inhalation parameter, an inhalation representation. The method comprises outputting, via the interface, the inhalation representation.

The disclosed electronic device, related method, and system may provide improved characterization and/or monitoring of an inhalation and/or an exhalation with an inhaler device with improved accuracy and precision. In other words, the present disclosure may provide improved audio-based characterization and/or monitoring of an inhalation and/or an exhalation with an inhaler device with improved accuracy and precision. It may be appreciated that the present disclosure may provide improved feedback on an inhalation and/or an exhalation with an inhaler device, the feedback being more intelligible for the user. The present disclosure may provide an improved prediction of inhalation parameters, such as an improved prediction of an inhalation flow when using an inhaler device. For example, by providing the inhalation representation the present disclosure may improve the visualization and/or the intelligibility to a user of an inhalation and/or an exhalation that the user has performed with an inhaler device. The inhalation representation may therefore provide information about an inhalation performance e.g., based on an inhalation flow and/or an inhalation time. In turn, the present disclosure may provide a faster and more customized feedback to a user after an inhalation and/or an exhalation with an inhaler device.

It may be appreciated that the present disclosure provides characterization and/or monitoring of inhalations and/or exhalations with inhaler devices, for example to ensure correct dosing of a medicament when using an inhaler device and track adherence of a user by providing the inhalation representation. The adherence of a user may be tracked e.g., over a week, a month, and/or a year.

An advantage of the present disclosure is that it is possible to directly interpret and/or determine the performance of the inhalations and/or exhalations with an inhaler device, based on one or more predicted inhalation parameters of an inhalation, for example including inhalation flow, inhalation duration, inhalation volume, and/or actuator coordination. Furthermore, the present disclosure provides the possibility to determine how and to what degree a medication dose was taken by the user, for instance by recognizing if the inhalation was performed by a person and recognizing if an inhaler container (such as capsule) is emptied. It may be appreciated that it is possible to recognize whether an inhalation was shallow or deep, continuous or interrupted, smooth or fluctuating, increasing or decreasing, based on the inhalation data, for example based on an inhalation flow pattern. Further it is possible to recognize the likelihood that a medication dose was outputted (such as emitted from an inhaler device), inhaled by a user, and/or deposited (e.g., in the lungs of the user).

Further, an advantage of the present disclosure is that the electronic device and the system are more versatile and may be used by any user taking medication with an inhaler device without the need for a healthcare professional monitoring the inhalation of the user. For example, the present disclosure may provide for training of a user, e.g., by instructing and/or guiding the user through an inhalation. This may for example be useful when a user starts using a new type of inhaler device.

Usually, users are taking the medication with an inhaler device alone at home without help or monitoring from a health care professional. Many patients also do not get proper training when they start using an inhaler device and/or change to a new type of inhaler device. It may be appreciated that the present disclosure provides for remote monitoring of a user of an inhaler device (such as a patient) by a healthcare professional. This enables a more quantitative and informative management for the healthcare professional of their patients and provides more empowerment to patients to take their medication correctly.

Another advantage of the present disclosure is that by using inhalation data indicative of an audio signal (e.g., sound-based) to assess inhalation flow compared with using electronic sensors is that it may be possible to obtain a very high sampling rate and temporal resolution of the measurements with sound. An electronic sensor may for example comprise an electronic flow meter, such as one or more of a cup anemometer, a pitot tube flow meter, a hot wire flow meter, and a vane flow meter. An electronic flow sensor may be seen as an electronic sensor with an air flow meter. For example, it may be possible to obtain a large number of inhalation flow values per second which can be useful in evaluating dynamic parameters in an inhalation, where the inhalation flow rate can change a lot from one fraction of a second to the next fraction. Further, it may be appreciated that by using inhalation data indicative of an audio signal (e.g., sound-based), the prediction model may be improved over time. This is not possible with inhalation flow rate measurements using non-acoustic sensors. Another advantage, is that inhalation data indicative of an audio signal (e.g., sound-based) may also capture unexpected events (something happening in the background of the inhalation) which may also be used to troubleshoot an unsuccessful measurement and/or be used for root cause analysis on a defect of the inhaler device, the prediction model, and/or a microphone of the electronic device.

Further, an advantage of the present disclosure is that the electronic device is more versatile and may be able to characterize and/or monitor an inhalation and/or exhalation performed with any inhaler device.

### BRIEF DESCRIPTION OF THE DRAWINGS

The above and other features and advantages of the present disclosure will become readily apparent to those skilled in the art by the following detailed description of examples thereof with reference to the attached drawings, in which:

FIG. 1 schematically illustrates an exemplary system for characterizing and/or monitoring an inhalation and/or an exhalation with an inhaler device according to the present disclosure, comprising an inhaler device and an electronic device according to the present disclosure,

FIGS. 2A-B are flow diagrams of an exemplary method according to the present disclosure,

FIG. 3 shows an example representation of inhalation data according to the present disclosure,

FIGS. 4A-B show an example representation of inhalation data according to the present disclosure,

FIGS. 5A-5B show an example scenario of characterization and/or monitoring of an inhalation and/or an exhalation with an inhaler device where the technique disclosed herein is applied,

FIGS. 6A-6B show an example scenario of characterization and/or monitoring of an inhalation and/or an exhalation with an inhaler device where the technique disclosed herein is applied,

FIGS. 7A-7B show an example scenario of characterization and/or monitoring of an inhalation and/or an exhalation with an inhaler device where the technique disclosed herein is applied,

FIGS. 8A-8B show an example scenario of characterization and/or monitoring of an inhalation and/or an exhalation with an inhaler device where the technique disclosed herein is applied,

FIGS. 9A-9B show an example scenario of characterization and/or monitoring of an inhalation and/or an exhalation with an inhaler device where the technique disclosed herein is applied,

FIG. 10 shows an example scenario of characterization and/or monitoring of an inhalation and/or an exhalation with an inhaler device where the technique disclosed herein is applied, and

FIGS. 11A-11B show an example scenario of characterization and/or monitoring of an inhalation and/or an exhalation with an inhaler device where the technique disclosed herein is applied.

### DETAILED DESCRIPTION

Various examples and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the examples. They are not intended as an exhaustive description of the disclosure or as a limitation on the scope of the disclosure. In addition, an illustrated example needs not have all the aspects or advantages shown. An aspect or an advantage described in conjunction with a particular example is not necessarily limited to that example and can be practiced in any other examples even if not so illustrated, or if not so explicitly described.

The figures are schematic and simplified for clarity, and they merely show details which aid understanding the disclosure, while other details have been left out. Throughout, the same reference numerals are used for identical or corresponding parts.

An electronic device for characterizing and/or monitoring an inhalation and/or an exhalation with an inhaler device is disclosed. In other words, the electronic device may be configured to characterize and/or monitor an inhalation and/or an exhalation with an inhaler device, such as an inhalation and/or an exhalation performed with the inhaler device. In other words, the electronic device may be configured to characterize and/or monitor an inhalation and/or an exhalation performed by a user when using an inhaler device. The electronic device may be configured to characterize and/or monitor an operation performed with an inhaler device, such as an operation of the inhaler device by a user. An inhalation and/or an exhalation may be seen as an operation of the inhaler device.

An inhaler device may be seen as an inhaler device for inhaling medication. The inhaler device may be seen as a sound generating inhaler, such as an inhaler with acoustic amplifier. In other words, the inhaler device may be an acoustic inhaler device. The inhaler device may comprise an acoustic amplifier in the form of a whistle. The inhaler device may be configured to provide a flow-dependent sound frequency profile, a flow dependent sound amplitude profile, and/or a flow dependent sound energy profile.

The inhaler device may alternatively be an inhaler without acoustic amplifier. The inhaler device may comprise different type of inhaler devices, such as powder based inhalers (dry powder inhaler), gas-based inhalers (such as metered dose inhaler and/or propellant-based inhaler), and/or nebulizer atomization based inhalers. The inhaler device may further comprise a single dose inhaler or multidose inhaler. The inhaler device may comprise an add-on container (such as a capsule) comprising the medication and/or an integrated medication container (such as capsule and/or storage) integrated in the inhaler device.

In one or more example electronic devices, the electronic device is a user equipment device.

In one or more example electronic devices, the electronic device is a server device.

The electronic device may comprise a user equipment device and/or a server device. The electronic device may be configured to operate on a user equipment device and/or a server device. In other words, the electronic device may be configured to act as a server device and/or a user equipment device. A user equipment device may for example be or comprise a mobile phone, such as a smartphone, a smart-watch, smart-speakers, a tablet, a computer, such as a laptop computer or PC, or a tablet computer. In other words, the electronic device may for example be a user device, such as a mobile phone or a computer, configured to perform a characterization and/or monitoring of an inhalation and/or an exhalation with an inhaler device. A server device may be configured on a cloud, such as a cloud network. Different operations configured to be performed by the electronic device and/or the system as disclosed herein may be performed at different devices, such as at the electronic device and/or at the server device.

The electronic device comprises a memory, an interface and one or more processors comprising predictor circuitry configured to operate according to a prediction model. In other words, the electronic device comprises one or more processors comprising a predictor engine configured to operate according to a prediction model.

The prediction model may for example comprise or make use of a neural network, artificial intelligence, deep learning, and/or machine learning.

In one or more example electronic devices, the prediction model comprises model layers including an input layer, one or more intermediate layers, and an output layer for provision of the predicted inhalation parameter. In one or more example electronic devices, the prediction model may be seen as a machine learning model. In one or more example electronic devices, the prediction model comprises a neural network. In one or more example electronic devices, the prediction model comprises neural network layers including an input layer, one or more intermediate layers, and an output layer for provision of the predicted inhalation parameter. In other words, the input layer, the one or more intermediate layers, and/or the output layer may be seen as layers of a machine learning model such as layers of a neural network. The one or more intermediate layers may be considered as hidden layers (such as hidden features). The one or more intermediate layers may include a first intermediate layer.

A model as referred to herein (such as the prediction model) may be seen as a model and/or a scheme and/or a mechanism and/or a method configured to provide, based on operational data (such as an audio signal and/or the inhalation data) and/or a previous model, one or more predicted inhalation parameters. A model as referred to herein (such as the prediction model) may be based on the same model architecture. A model architecture may be based on a neural network, such as comprising one or more different type of layers and/or number of layers. A model architecture may be seen as configuration of a model, such as comprising one or more parameters of a model.

In one or more example electronic devices, the model as referred to herein may be stored on a non-transitory storage medium (for example, on the memory of the electronic device). The model may be stored on a non-transitory storage medium of the electronic device being configured to execute the model. In one or more example electronic devices, the model may comprise model data and or computer readable instructions (for example based on inhalation data and/or audio signal, such as historical inhalation data). The model data and/or the computer readable instructions may be used by the electronic device and/or the server device. The model (such as model data and/or the computer readable instructions) may be used by the server device and/or the electronic device to determine predicted inhalation parameters and inhalation representations. In other words, the model (such as model data and/or the computer readable instructions) may be used by the server device and/or the electronic device to determine one or more parameters and/or features as described herein, such as inhalation and/or exhalation parameter and/or features.

In one or more example electronic devices, the predictor circuitry comprises a neural network module configured to operate according to a neural network.

In one or more example electronic devices, the neural network is a deep neural network, such as a convolutional neural network and/or a recurrent neural network. For example, the neural network may comprise a one dimensional convolutional neural network or a two dimensional convolutional neural network.

In one or more example electronic devices, the predictor circuitry comprises a regressor module configured to operate according to a regression model.

The prediction model may be based on a neural network (such as a convolutional neural network, a deep learning neural network, a recurrent neural network, and/or a combined learning circuitry). The predictor circuitry may be configured to determine (and optionally identify) one or more patterns in existing data (inhalation data, audio signal(s), sound patterns (such as inhalation patterns), and/or predicted inhalation parameters) in order to facilitate making predictions for subsequent predicted inhalation parameters. For example, the prediction circuitry may be configured to determine (such as recognize) an inhalation pattern based on plot of peak sound frequency over time. Additional prediction models may be generated to provide substantially reliable predictions of inhalation parameters of a prediction of an inhalation flow.

The predictor circuitry (such as the neural network module and/or the regressor module) may be configured to operate according to a machine learning scheme configured to determine a rule or a pattern or a relation that maps inputs to outputs, so that when subsequent novel inputs are provided the predictor circuitry may, based upon the rule, pattern or relation, accurately predict the correct output. In one or more embodiments, the prediction model may first extract one or more features from input inhalation data, such as by using signal processing methods (such as filters), statistics of the signals (such as mean, max, median, and/or quantile), and/or results from unsupervised learning methods (such as dimension reduction methods, clustering, and/or auto-encoder). The one or more features may then be fed into a regression and/or classification model that is trained using machine learning techniques.

In one or more example electronic devices, the processor is configured to train and/or update the prediction model based on one or more of: the inhalation data and the predicted inhalation parameter. In one or more embodiments, the processor may be configured to train and/or update the prediction model based on the outcome of the inhalation representation (for example, by comparing the predicted inhalation parameter and known inhalation parameters). The prediction model that the predictor circuitry operates according to, may be trained and/or updated (such as retrained or finetuned). The training of the prediction model may be a supervised learning setup, where the inhalation data in the input data and the network quality data can be labelled. The prediction model or changes to the prediction model may be based on new data, such as new sensor data, and/or new prediction data.

The processor is configured to obtain inhalation data (such as inhalation data and/or exhalation data), where the inhalation data is indicative of an audio signal (such as audio data) representing an inhalation and/or an exhalation with the inhaler device. In other words, the inhalation data may be based on an audio signal representing an inhalation and/or an exhalation with the inhaler device. For example, the inhalation data may be based on an audio signal from an operation, such as an inhalation and/or an exhalation operation and/or procedure, performed by a user with the inhaler device. The user may be a user of the inhaler device taking a medication dose with the inhaler device. To obtain inhalation data may comprise that the processor is configured to determine, retrieve, generate, and/or receive the inhalation data. The inhalation data may be seen as and/or based on an audio recording of an operation, such as an inhalation and/or an exhalation, performed by a user with the inhaler device. In other words, the inhalation data may be seen as and/or based on an audio recording of a sound sequence of an operation performed by a user with the inhaler device, such as a sound produced by an inhalation, an exhalation, and/or an amplifier of the inhaler device. The inhalation data may be based on sound data obtained by the electronic device, such as using the processor, from the inhaler device.

In one or more example electronic devices, the audio signal has a flow dependent sound frequency profile. In other words, the inhaler device may be configured to generate a flow-dependent sound frequency profile that the audio signal is based on. For example, the inhaler device, such as acoustic inhaler device, may be configured to generate an audio signal having a flow dependent sound frequency profile. The inhaler device may comprise an acoustic amplifier, such as a whistle, configured to generate an audio signal comprising a flow-dependent sound frequency profile. For example, the inhaler device comprises a hole tone whistle and/or a corrugated pipe whistle configured to generate an audio signal comprising a flow-dependent sound frequency profile. In other words, the audio signal, such the sound produced by the inhaler device, may comprise a sound signature when performing an inhalation and/or an exhalation with the inhaler device. For example, the audio signal, such the sound produced by the inhaler device, may comprise a distinct sound frequency profile for each level of air flow when using the inhaler device. It may be appreciated that the higher the inhalation flow is the louder (amplitude) the sound produced by an inhaler device becomes. This can also be translated as sound energy. For example, by using an inhaler device comprising an acoustic amplifier (such as a whistle) having a substantially linear relationship between sound frequency and flow rate the electronic device, such as using the predictor circuitry, may determine a predicted inhalation parameter indicative of an inhalation flow with the inhaler device, e.g., based on the sound frequency profile of the audio signal generated by the inhaler device.

By using the sound frequency sound profile, a more robust prediction of inhalation flow may be achieved as it is independent of distance from the inhaler device.

This is an advantage compared to inhaler devices having an acoustic amplifier being harmonic devices, or other types of inhaler devices that do now show such a clear relationship between sound frequency and flow rate.

In one or more example electronic devices, the electronic device comprises one or more microphones for obtaining the audio signal. For example, the electronic device may comprise a mobile phone comprising one or more microphones for obtaining the audio signal. In other words, the electronic device comprises one or more microphones configured to generate and/or provide the audio signal based on a sound generated by the inhaler device, such as the acoustic inhaler device. The one or more microphones may have a sampling rate of at least 50 KHz. By having a sampling rate of at least 50 kHz it may ensure that sound from the inhaler device is captured at high resolution and that an electronic device according to the present disclosure, e.g., with a sound-based inhalation flow meter, provides a high temporal resolution for the predicted inhalation parameter. This is an advantage because of the high sampling rate and continuity of the inhalation data, e.g., compared with sensor based technology.

The processor is configured to determine, based on the inhalation data, using the predictor circuitry, a predicted inhalation parameter indicative of a prediction of an inhalation flow with the inhaler device. A predicted inhalation parameter may be seen as a predicted physiological factor indicative of a prediction of an inhalation and/or an exhalation with the inhaler device. An inhalation flow as disclosed herein may be seen as an inhalation flow rate. In other words, the processor is configured to determine, based on the inhalation data, using the prediction model, a predicted inhalation parameter indicative of a prediction of an inhalation flow with the inhaler device. In one or more example electronic devices, the processor is configured to determine, based on the inhalation data, using the predictor circuitry, a predicted inhalation parameter indicative of an inhalation flow and/or an exhalation flow. Determining a predicted inhalation parameter indicative of a prediction of an inhalation flow may comprise extracting one or more inhalation and/or exhalation features from the audio signal and/or the inhalation data. In other words, determining a predicted inhalation parameter indicative of a prediction of an inhalation flow may be based on one or more features extracted from the audio signal and/or the inhalation data. In one or more example electronic devices, the processor is configured to determine the predicted inhalation parameter based on one or more features extracted from the audio signal and/or the inhalation data. For example, when the audio signal has a flow dependent sound frequency, the processor may be configured to extract a sound frequency from the audio signal, and to determine an inhalation flow and/or an exhalation flow based on the extracted sound frequency. In other words, the audio signal may have a flow dependent sound frequency profile (e.g., sound spectral profile) and the processor may be configured to extract one or more sound frequencies, such as one or more sound frequency peaks, from the audio signal, and to determine a predicted inhalation parameter, such as an inhalation flow and/or an exhalation flow, based on the extracted one or more sound frequencies. For example, the inhalation data, such as the audio data, may comprise for each inhalation and/or exhalation multiple sound frequency peaks.

The one or more inhalation and/or exhalation features may for example comprise one or more of: an inhalation and/or exhalation phase (such as a phase of an operation of the user when taking medication with the inhaler device), an inhalation flow feature, an amplitude feature, a time feature (such as duration feature), an inhalation volume feature, a flow feature, and a flow acceleration feature. For example, the time feature may be indicative of one or more of: a gap in an inhalation, an intermittent inhalation, and a continuity of inhalation (e.g., whether an inhalation is continuous or not). An inhalation and/or exhalation phase may comprise one or more of: an introductory inhalation phase, an intermediate inhalation phase, an ending inhalation phase, and an exhalation phase.

An exhalation feature may comprise one or more exhalation peak flows. The exhalation and the inhalation may be unrelated. For example, it may be advantageous that the user of the inhaler device holds the breath for a few seconds to ensure that the medication dose (such as drug) deposits in the lungs of the user, and then after that the user may take the mouth off from the inhaler device and exhale into the air. An exhalation feature may therefore comprise a timestamp for the exhalation, an exhalation duration, and/or a duration between the inhalation and the start of the exhalation (e.g., the duration where the user holds the breath). The determination of a predicted inhalation parameter may comprise to determine an exhalation feature. An exhalation feature may be determined based inhalation data representative of an exhalation peak flow test, e.g., for testing a lung performance of a user.

In one or more example electronic devices, the obtaining of the inhalation data comprises to obtain, based on the audio signal, sound data of the audio signal, and wherein the determination of the predicted inhalation parameter is based on the sound data. In one or more example electronic devices, the processor is configured to obtain, based on the audio signal, sound data of the audio signal, e.g., from the inhaler device. In one or more example electronic devices, the processor is configured to determine the predicted inhalation parameter based on the sound data. In other words, the processor may be configured to extract one or more audio features from the audio signal, where the sound data comprises the one or more audio features. It may be appreciated that the prediction model may be configured to extract one or more audio features from the audio signal to obtain (such as provide) the sound data. The sound data may comprise one or more audio features, such as a sound frequency feature (such as spectrum of sound frequencies for each point in time), an amplitude feature, and/or a time feature. In other words, the sound data may comprise a sound frequency profile over time, and/or an amplitude profile over time. The sound frequency may for example be in the range of 0 to 25 KHz.

In one or more example electronic devices, the obtaining of the inhalation data comprises to apply one or more filters to the audio signal. In one or more example electronic devices, the processor may be configured to apply one or more filters to the audio signal to obtain, such as determine, the inhalation data. In other words, the processor comprises a pre-processing module configured to apply one or more filters to the audio signal. To apply one or more filters may comprise to apply a low-pass filter and/or a high-pass filter to the audio signal. By applying one or more filters to the audio signal the signal to noise ratio (SNR) may be enhanced. In one or more example electronic devices, the prediction circuitry is configured to apply the one or more filters by using the prediction model on the audio signal. In other words, the prediction model may be configured to extract the audio signal representing an inhalation and/or an exhalation with an inhaler device from an audio signal (such as audio data) by applying one or more filters.

In one or more example electronic devices, the obtaining of the inhalation data comprises to identify a background noise (such as background sounds) from the audio signal. In one or more example electronic devices, the processor may be configured to identify a background noise from the audio signal. In one or more example electronic devices, the prediction circuitry is configured to identify a background noise by using the prediction model on the audio signal. For example, the prediction model may be pre-trained based on a plurality of background noises in order to be able to identify one or more background noises from the audio signal. In other words, the prediction model may be trained to extract the audio signal representing an inhalation and/or an exhalation with an inhaler device from an audio signal (such as audio data) comprising one or more background noises (such as background sounds).

In one or more example electronic devices, the obtaining of inhalation data comprises to split (such as cut) the audio signal (such as audio data) into a plurality of audio samples. In one or more example electronic devices, the processor is configured to split the audio signal into a plurality of audio samples. For example, the processor may comprise an audio processing module configured to process the audio signal, such as configured to split the audio signal into a plurality of audio samples. In other words, by splitting the audio signal (such as audio data) into a plurality of audio samples it may be possible to divide the audio signal into different phases of the inhalation and/or exhalation.

In one or more example electronic devices, the determination of the predicted inhalation parameter is based on one or more audio samples of the plurality of audio samples. In one or more example electronic devices, the processor is configured to determine the predicted inhalation parameter based on one or more audio samples of the plurality of audio samples. In other words, the predictor circuitry may be configured to provide one or more audio samples of the plurality of audio samples as an input to the prediction model. In other words, the processor (such as the predictor circuitry) may be configured to provide the plurality of the audio samples as an input to the prediction model and keep the structure of the audio signal, such as keep the chronology of the audio samples.

To split the audio signal (such as audio data) into a plurality of audio samples may comprise to split the audio signal at a sample rate in the range of 1 ms to 1000 ms, such as in the range of 5 ms to 100 ms. By splitting the audio signal into a plurality of audio samples, it may be possible to balance between capturing more details about the inhalation and/or the exhalation with shorter audio samples when determining the predicted inhalation parameter, and flattening out the data by averaging out the audio signal over fewer audio samples, such as averaging out an inhalation flow profile over fewer sample points, when determining the predicted inhalation parameter. For example, by splitting the audio data into short audio samples in the range of 1 ms to 10 ms it may be possible to determine a predicted inhalation parameter representative of a specific period of the inhalation and/or exhalation, such as predicted inhalation parameter representative of a specific event of the inhalation and/or exhalation. By using audio samples in the range of 1 ms to 1000 ms, it may be possible to reduce the risk of confusion for the prediction model since the audio samples are representative of substantially one event of the inhalation and/or exhalation.

In one or more example electronic devices, the obtaining of inhalation data comprises to shuffle the plurality of audio samples. In one or more example electronic devices, the processor is configured to shuffle the plurality of audio samples. In one or more example electronic devices, the determination of the predicted inhalation parameter is based on the shuffled audio samples.

In one or more example electronic devices, the processor is configured to determine the predicted inhalation parameter based on the shuffled audio samples. In other words, the predictor circuitry may be configured to provide one or more of the shuffled audio samples of the plurality of audio samples as an input to the prediction model. In other words, the processor (such as the predictor circuitry) may be configured to provide the plurality of shuffled audio samples as an input to the prediction model. By providing the plurality of shuffled audio samples as an input to the prediction model the prediction model may be trained to determine the predicted inhalation parameter without having the structure and/or chronology of the audio samples. This may provide a more robust prediction model to determine predicted inhalation parameters. Further, by splitting (such as cutting) and/or shuffling the audio signal, more audio data is provided, for example to train the prediction model, expand the audio data library, and/or determine predicted inhalation parameters on smaller audio samples. It may be appreciated that it may be possible to determine a plurality of predicted inhalation parameters based on the same inhalation (such as inhalation operation).

In one or more example electronic devices, the obtaining of inhalation data comprises to transform the audio signal to a spectrogram. In one or more example electronic devices, the processor is configured to transform the audio signal to a spectrogram.

In one or more example electronic devices, the determination of the predicted inhalation parameter is based on the spectrogram. In one or more example electronic devices, the processor is configured to determine the predicted inhalation parameter based on the spectrogram.

In one or more example electronic devices, the obtaining of inhalation data comprises to transform the audio signal to a spectrogram and to split the spectrogram into a plurality of spectrogram samples. In one or more example electronic devices, the determination of the predicted inhalation parameter is based on one or more of the plurality of spectrogram samples.

In one or more example electronic devices, the obtaining of inhalation data comprises to transform the audio signal to a spectrogram, to split the spectrogram into a plurality of spectrogram samples, and to shuffle the plurality of spectrogram samples. In one or more example electronic devices, the determination of the predicted inhalation parameter is based on the plurality of shuffled spectrogram samples. By transforming the audio signal to a spectrogram it may be possible to classify one or more events of the inhalation and/or exhalation by using the prediction model.

In one or more example electronic devices, the obtaining of the inhalation data comprises to obtain image data, such as based on the audio signal. In one or more example electronic devices, the processor is configured to obtain image data. In one or more example electronic devices, the determination of the predicted inhalation parameter is based on the image data. In one or more example electronic devices, the processor is configured to determine the predicted inhalation parameter based on the image data. In other words, the processor is configured to use the image data as input to the prediction model. In one or more example electronic devices, the processor is configured to determine one or more image features based on the image data. The one or more image features may be indicative of one or more of: an inhalation and/or exhalation phase (such as a phase of an operation of the user when taking medication with the inhaler device), an inhalation flow, an amplitude, time (such as duration), an inhalation volume, and an inhalation flow acceleration.

In one or more example electronic devices, the processor is configured to determine the predicted inhalation parameter based on the one or more features.

The image data may comprise an image indicative of a sound frequency of the inhalation and/or exhalation. In other words, the image data may for example comprise a plot, such as a graph, e.g., a two dimensional plot, with one or more of the sound frequency, the amplitude, and the energy on the y-axis and the time on the x-axis. For example, the image data may comprise a time lapse of an inhalation and/exhalation. A spectrogram as described above may be seen as image data, such as the spectrogram having the format of an image.

In one or more example electronic devices, the determination of the predicted inhalation parameter comprises to determine inhalation flow data. Inhalation flow data may for example comprise one or more of the features as described above, such as the one or more extracted features and/or the one or more inhalation and/or exhalation features. The determination of the inhalation flow data may comprise to determine a predicted inhalation flow, such as a predicted inhalation flow over time.

In one or more example electronic devices, the processor is configured to determine, based on the predicted inhalation parameter, one or more of: a duration of inhalation, an inhalation volume, an average inhalation flow, a maximum inhalation flow, a minimum inhalation flow, median inhalation flow, an inhalation flow acceleration, and one or more exhalation parameters. In other words, the predicted inhalation parameter may comprise one or more of: a duration of inhalation, an inhalation volume, an average inhalation flow, a maximum inhalation flow, a minimum inhalation flow, a median inhalation flow, an inhalation flow acceleration, and one or more exhalation parameters. In other words, an output of the prediction model may comprise one or more of: a duration of inhalation, an inhalation volume, an average inhalation flow, a maximum inhalation flow, a minimum inhalation flow, median inhalation flow, an inhalation flow acceleration, and one or more exhalation parameters. In one or more example electronic devices, the processor is configured to determine, based on the inhalation data and/or the audio signal, one or more of: a duration of inhalation, an inhalation volume, an average inhalation flow, a maximum inhalation flow, a minimum inhalation flow, median inhalation flow, an inhalation flow acceleration, and one or more exhalation parameters. An inhalation duration may for example be in the range of 1 s to 10 s. A successful inhalation may be of at least 1 s, at least 2 s, at least 3 s, at least 4 s, at least 5 s, or at least 6 s. An inhalation duration for a successful inhalation may depend on the type of inhaler device. An inhalation flow (such as flow rate) may be in the range of 1 l/min to 100 l/min, such as in the range of 5 l/min to 90 l/min, and/or in the range of 10 l/min to 90 l/min.

An exhalation parameter may comprise one or more of: an exhalation flow, a duration of exhalation, an exhalation volume, an average exhalation flow, a maximum exhalation flow, an exhalation inhalation flow, median exhalation flow, and an exhalation flow acceleration.

The processor is configured to determine, based on the predicted inhalation parameter, an inhalation representation. The inhalation representation may be seen as a representation indicative of all or part of the inhalation and/or exhalation performed by a user with the inhaler device. In other words, the inhalation representation may be seen as an inhaler device operation representation. The inhalation representation may be seen as and/or comprise an evaluation of an inhalation and/or an exhalation with the inhaler device, such as an inhalation evaluation. In other words, the inhalation representation may be seen as and/or comprise an evaluation of an operation of a user when taking a medication with the inhaler device. In one or more example electronic devices, the inhalation representation comprises an inhalation and/or exhalation score indicative of a performance of a user when performing an inhalation and/or exhalation with the inhaler device, such as a performance of a medication intake. The inhalation representation may indicate whether an inhalation, an exhalation, and/or a medication intake was successful or not. For example, the score may be indicative of a successful inhalation, exhalation, and/or medication intake when the predicted inhalation parameter satisfies a criterion. For example, the processor may be configured to determine whether the score satisfies a criterion (such as the first criterion). When the score is above or equal to a threshold (such as the first threshold), it may be determined that the score satisfies the criterion (such as first criterion).

In one or more example electronic devices, the inhalation representation comprises a representation of the predicted inhalation parameter. For example, the inhalation representation may comprise a representation of an inhalation flow with the inhaler device, such as a graph or a plot of the inhalation flow over time, a spectrogram and/or the image data as described herein. The inhalation representation may comprise one or more of: a representation of a duration of inhalation, a representation of an inhalation volume, a representation of an inhalation flow profile, a representation of an average inhalation flow, a representation of a maximum inhalation flow, a representation of a minimum inhalation flow, a representation of a median inhalation flow, a representation of an inhalation flow acceleration, and a representation of one or more exhalation parameters. The inhalation representation may be indicative of the whole inhalation and/or exhalation with the inhaler device, and/or part of the inhalation and/or exhalation with the inhaler device, such as a phase of the inhalation and/or exhalation with the inhaler device.

In one or more example electronic devices, the inhalation representation is indicative of an evaluation of an inhaler device. In other words, the processor may be configured to determine a state of the inhaler device based on the inhalation data and/or the audio signal.

An advantage of having an inhalation representation, e.g. after an inhalation, an exhalation, and/or an operation with the inhaler device has been performed, may be that the user of the electronic device and/or the inhaler device may see or be informed right after the inhalation, such as operation, about his/her performance or the outcome of the inhalation in relation to the predicted inhalation parameter. Therefore, when an inhalation was unsuccessful the user may be aware of it and may be able to repeat the inhalation and/or operation and/or improve his/her performance for the next inhalation and/or operation. Furthermore, the user of the electronic device and/or the inhaler device may get a better feedback on his/her performance or on the outcome of the inhalation and/or operation. The inhalation representation may provide a gamification of the users' performances. The inhalation representation may for example increase the intelligibility of the feedback to the user, e.g. by being able to visualize an inhalation and/or operation with the inhaler device, and further to improve his/her inhalation technique by being able to visualize an improvement of performances.

Optionally, the processor is configured to output, via the interface, the inhalation representation.

In one or more example electronic devices, outputting the inhalation representation may comprise outputting, via the interface of the electronic device, the inhalation representation. Outputting the inhalation representation may comprise displaying a user interface indicative of the inhalation representation. In one or more exemplary methods, outputting the inhalation representation may comprise outputting, via the interface of the electronic device, a first inhalation representation, a second inhalation representation, a third inhalation representation, etc.

Outputting the inhalation representation may comprise displaying a user interface indicative of the inhalation representation.

A user interface may comprise one or more, such as a plurality of, user interface objects. For example, the user interface may comprise one or more user interface objects, such as a first user interface object and/or a second user interface object. A user interface object may refer herein to a graphical representation of an object that is displayed on an interface of the electronic device, such as a display. The user interface object may be user-interactive, or selectable by a user input. For example, an image (e.g., icon), a button, and text (e.g., hyperlink) each optionally constituting a user interface object. The user interface object may form part of a widget. A widget may be seen as a mini-application that may be used by the user. To output the inhalation representation may comprise to output an inhalation representation comprising one or more of text (such as a text string) and/or a phrase, a score (such as an evaluation score and/or an inhalation and/or exhalation score), image data (such as one or more images), a spectrogram, and/or a user interface object comprising one or more of the previous. For example, to output the inhalation representation may comprise to output an inhalation representation comprising a spectrogram of an inhalation flow profile over time as disclosed herein and an indication of where on the spectrogram the user may improve his/her inhalation and/or exhalation. For example, to output the inhalation representation may comprise to output an inhalation representation comprising a score, such as an evaluation score of the inhalation and/or exhalation of the user with the inhaler device.

In one or more example electronic devices, the processor is configured to determine whether the predicted inhalation parameter satisfies a first criterion. In other words, the processor may be configured to determine whether the predicted inhalation parameter is above, below, or equal to an inhalation flow threshold and/or is within a certain range indicative of the first criterion. In other words, the predicted inhalation parameter may satisfy the first criterion when the predicted inhalation parameter is above or equal to a first threshold. For example, the predicted inhalation parameter may satisfy the first criterion when the inhalation flow is above or equal to a first threshold. In other words, when the predicted inhalation parameter satisfies the first criterion an inhalation, exhalation, and/or operation has been determined to be successful. Formulated differently, when the predicted inhalation parameter satisfies the first criterion a medication intake by inhalation with the inhaler device has been determined to be successful.

In one or more example electronic devices, the processor is configured to determine whether one or more of the following satisfy the first criterion: an inhalation and/or exhalation phase (such as a phase of an operation of the user when taking medication with the inhaler device), an inhalation flow feature, an amplitude feature, a time feature (such as duration feature), an inhalation volume feature, a flow feature, and a flow acceleration feature.

When it is determined that the predicted inhalation parameter does not satisfy the first criterion, the predicted inhalation parameter may be below the first threshold and/or outside a certain range indicative of the first criterion. When it is determined that the predicted inhalation parameter does not satisfy the first criterion, an inhalation, exhalation, and/or operation is determined to be unsuccessful. Formulated differently, when the predicted inhalation parameter does not satisfy the first criterion a medication intake by inhalation with the inhaler device has been determined to be unsuccessful.

An inhalation flow threshold, such as the first threshold, may comprise a pre-configured threshold, e.g., based on one or more of an optimum inhalation flow value or range, a user's profile, and/or a previous performance of a user.

In one or more example electronic devices, the first criterion comprises a first threshold associated with a first inhalation flow and/or a first inhalation time. In other words, the processor may be configured to determine whether the predicted inhalation parameter is above, below, or equal a first inhalation flow threshold and/or a first inhalation time threshold and/or is within a certain range of the first inhalation flow value and/or range and/or the first inhalation time value and/or range. In other words, the predicted inhalation parameter may satisfy the first criterion when the predicted inhalation parameter is above or equal to the first inhalation flow threshold and/or the first inhalation time threshold. For example, the predicted inhalation parameter may satisfy the first criterion when the inhalation flow is above or equal to the first inhalation flow threshold and/or an inhalation time is above, equal to, and/or within a range of the first inhalation time threshold.

In one or more example electronic devices, in accordance with the predicted inhalation parameter satisfying the first criterion, the processor is configured to determine a first recommendation. The first recommendation may be seen as a feedback to the user of the inhaler device regarding an inhalation and/or exhalation (such as an operation) with the inhaler device. The first recommendation may be seen as a first evaluation. The first recommendation may be indicative of an inhalation, an exhalation, and/or a medication intake that was successful or partly successful. The first recommendation may be seen as and/or comprise an advisory action that the user of the inhaler device should do. The first recommendation may for example comprise text (such as a message to the user) and/or phrases such as: “The medication intake was successful”, “Continue with your inhalation technique”, “Your inhalation was successful”, and/or “Your inhalation was successful, but the medication container of the inhaler device is soon to be empty. Please verify the container status of the medication container of the inhaler device”.

In one or more example electronic devices, the processor is configured to output, via the interface (such as the interface of the electronic device), the first recommendation. In other words, the processor may be configured to output the first recommendation in the form of a text (such as a message to a user) and/or a phrase, an evaluation score, image data (such as one or more images), a spectrogram, and/or a user interface as described herein.

In one or more example electronic devices, the first recommendation is comprised in the inhalation representation. In other words, the processor may be configured to include the first recommendation in the inhalation representation. For example, the processor may be configured to output an inhalation representation comprising the first recommendation.

In one or more example electronic devices, the first recommendation comprises one or more of: an inhalation depth recommendation, an inhalation duration recommendation, an inhalation flow recommendation, and an inhalation preparation recommendation.

In one or more example electronic devices, the second recommendation comprises one or more of: an inhalation depth recommendation, an inhalation duration recommendation, an inhalation flow recommendation, and an inhalation preparation recommendation.

An inhalation depth recommendation may be seen as a recommendation to the user of the inhaler device regarding the inhalation depth when performing the inhalation that the inhalation data is based on. The inhalation depth recommendation may indicate whether the inhalation depth of the user of the inhaler device when performing the inhalation that the inhalation data is based on was satisfying or not. For example, an inhalation depth recommendation may comprise “Your inhalation depth was satisfying”, “Your inhalation depth was not satisfying, please inhale deeper next time”, “Your inhalation depth was not satisfying, next time please repeat the inhalation operation (such as medication intake) and inhale deeper”.

An inhalation duration recommendation may be seen as a recommendation to the user of the inhaler device regarding the inhalation duration when performing the inhalation that the inhalation data is based on. The inhalation duration recommendation may indicate whether the inhalation duration of the user of the inhaler device when performing the inhalation that the inhalation data is based on was satisfying or not. For example, an inhalation duration recommendation may comprise “Your inhalation duration was satisfying”, “Your inhalation duration was not satisfying, please inhale for a longer time next time”, or “Your inhalation duration was not satisfying, next time please repeat the inhalation operation (such as medication intake) and inhale for a longer time”.

An inhalation flow recommendation may be seen as a recommendation to the user of the inhaler device regarding the inhalation flow when performing the inhalation that the inhalation data is based on. The inhalation flow recommendation may indicate whether the inhalation flow of the user of the inhaler device when performing the inhalation that the inhalation data is based on was satisfying or not. The inhalation flow recommendation may comprise a minimum inhalation flow recommendation, e.g., being indicative of a minimum inhalation flow that is recommended to the user for achieving a successful medication intake. For example, an inhalation flow recommendation may comprise “Your inhalation flow was satisfying”, “Your inhalation flow was not satisfying, please inhale with a higher flow next time”, or “Your inhalation flow was not satisfying, next time please repeat the inhalation operation (such as medication intake) and inhale with a higher flow”.

An inhalation preparation recommendation may be seen as a recommendation to the user of the inhaler device regarding the inhalation preparation when performing the inhalation that the inhalation data is based on. An inhalation preparation may for example comprise that the user starts inhaling before actuating and/or activating a medication container of the inhaler device. In other words, an inhalation preparation may comprise to coordinate an inhalation and actuation and/or activating of a medication container of the inhaler device before and/or when starting a medication intake operation. An inhalation preparation may for example comprise that the user clears his/her throat before starting a medication intake procedure (such as inhalation).

The inhalation preparation recommendation may indicate whether the inhalation preparation of the user of the inhaler device when performing the inhalation that the inhalation data is based on was satisfying or not. For example, an inhalation preparation recommendation may comprise “Your inhalation preparation was satisfying”, “Your inhalation preparation was not satisfying, please start inhaling before actuating and/or activating a medication container of the inhaler device next time”, or “Your inhalation preparation was not satisfying, next time please repeat the inhalation operation (such as medication intake) and start inhaling before actuating and/or activating a medication container of the inhaler device”.

In one or more example electronic devices, in accordance with the predicted inhalation parameter not satisfying the first criterion, the processor is configured to determine a second recommendation. The second recommendation may be seen as a feedback to the user of the inhaler device regarding an inhalation and/or exhalation (such as an operation) with the inhaler device. The second recommendation may be seen as a second evaluation. The second recommendation may be indicative of an inhalation, an exhalation, and/or a medication intake that was unsuccessful or partly unsuccessful. The second recommendation may be seen as and/or comprise an advisory action that the user of the inhaler device should do. The second recommendation may for example comprise text (such as a message to the user) and/or phrases such as: “The medication intake was unsuccessful”, “Your inhalation technique needs to be improved”, “Your inhalation was unsuccessful”, “You exhaled too much when performing the medication intake”, “Your inhalation flow was satisfying, but the inhalation time was not sufficient”, and/or “Your inhalation was unsuccessful, because the medication container of the inhaler device was empty. Please change inhaler device or replace the medication container of the inhaler device”.

A system for characterizing and/or monitoring an inhalation and/or an exhalation with an inhaler device is disclosed. The system comprises the inhaler device and an electronic device according to the present disclosure.

A method, for characterizing and/or monitoring an inhalation and/or an exhalation with an inhaler device is disclosed. The method comprises obtaining inhalation data, where the inhalation data is indicative of an audio signal representing an inhalation and/or an exhalation with the inhaler device. The method comprises determining, based on the inhalation data, using the predictor circuitry, a predicted inhalation parameter indicative of a prediction of an inhalation flow with the inhaler device. The method comprises determining, based on the predicted inhalation parameter, an inhalation representation. The method comprises outputting, via the interface, the inhalation representation.

It is to be understood that a description of a feature in relation to the electronic device(s) is also applicable to the corresponding feature in the system(s), and/or the method(s) for characterizing and/or monitoring an inhalation and/or an exhalation with an inhaler device.

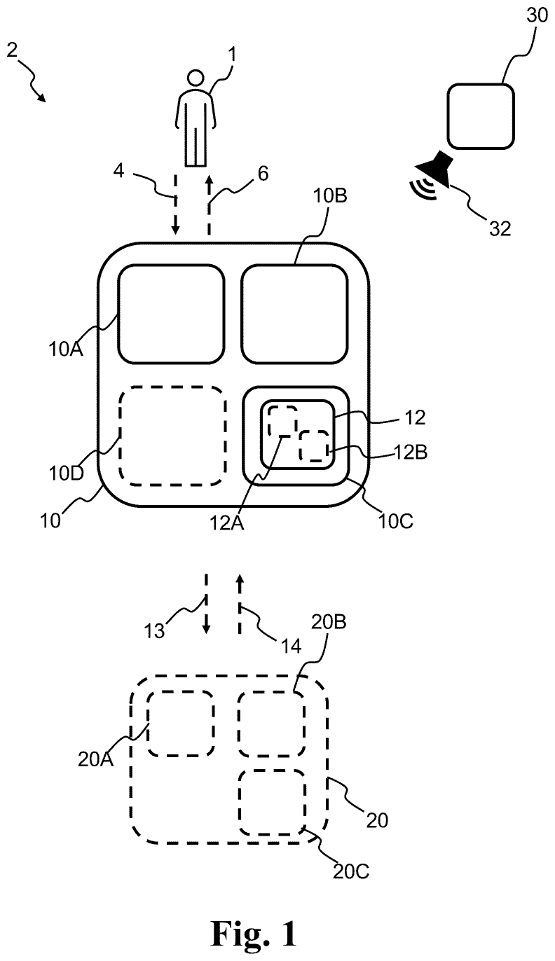

FIG. 1 schematically illustrates an exemplary system, such as a system 2 for characterizing and/or monitoring an inhalation and/or an exhalation with an inhaler device, according to the present disclosure. The system 2 comprises an inhaler device 30. The system 2 comprises an electronic device 10, such as the electronic device for characterizing and/or monitoring an inhalation and/or an exhalation with an inhaler device according to the present disclosure.

In other words, the electronic device 10 may be configured to characterize and/or monitor an inhalation and/or an exhalation with the inhaler device 30, such as an inhalation and/or an exhalation performed with the inhaler device 30. In other words, the electronic device 10 may be configured to characterize and/or monitor an inhalation and/or an exhalation performed by a user 1 when using the inhaler device 30. The electronic device 10 may be configured to characterize and/or monitor an operation performed with the inhaler device 30, such as an operation of the inhaler device 30 by the user 1. An inhalation and/or an exhalation may be seen as an operation of the inhaler device 30.

The electronic device 10 comprises a memory 10A, an interface 10B (such as one or more interfaces), and a processor 10C. Optionally, the system 2 comprises a server device 20. The server device 20 comprises a memory 20A, an interface 20B (such as one or more interfaces), and a processor 20C (such as one or more processors). The processor 10C comprises predictor circuitry 12 configured to operate according to a prediction model. In one or more example electronic devices and/or systems, the model as referred to herein may be stored on a non-transitory storage medium (for example, on the memory 10A of the electronic device 10, and/or on the memory 20A of the server device 20). In one or more example electronic devices and/or systems, the predictor circuitry 12 comprises a neural network module 12A configured to operate according to a neural network.

In one or more example electronic devices and/or systems, the neural network is a deep neural network, such as a convolutional neural network and/or a recurrent neural network. For example, the neural network may comprise a one dimensional convolutional neural network or a two dimensional convolutional neural network.

In one or more example electronic devices and/or systems, the predictor circuitry 12 comprises a regressor module 12B configured to operate according to a regression model.

In one or more example electronic devices and/or systems, the electronic device 10 is a user equipment device.

In one or more example electronic devices and/or systems, the electronic device 10 is a server device.

The electronic device 10 may comprise a user equipment device and/or a server device. The electronic device 10 may be configured to operate on a user equipment device and/or a server device. In other words, the electronic device 10 may be configured to act as a server device and/or a user equipment device. A user equipment device may for example be or comprise a mobile phone, such as a smartphone, a computer, such as a laptop computer or PC, or a tablet computer. In other words, the electronic device 10 may for example be a user device, such as a mobile phone or a computer, configured to perform a characterization and/or monitoring of an inhalation and/or an exhalation with the inhaler device 30. A server device (such as server device 20) may be configured on a cloud, such as a cloud network. Different operations configured to be performed by the electronic device 10 and/or the system 2 as disclosed herein may be performed at different devices, such as at the electronic device 10 and/or at the server device 20. In one or more example electronic devices and/or systems, when the electronic device 10 acts as a user equipment device, the system 2 may also comprise the server device 20.

The processor 10C is configured to obtain inhalation data (such as inhalation data and/or exhalation data), where the inhalation data is indicative of an audio signal (such as audio data) representing an inhalation and/or an exhalation with the inhaler device 30. In other words, the inhalation data may be based on an audio signal representing an inhalation and/or an exhalation with the inhaler device 30. For example, the inhalation data may be based on an audio signal from an operation, such as an inhalation and/or an exhalation operation and/or procedure, performed by the user 1 with the inhaler device 30. The user 1 may be a user of the inhaler device 30 taking a medication dose with the inhaler device 30. To obtain inhalation data may comprise that the processor is configured to determine, retrieve, generate, and/or receive the inhalation data. Optionally, the processor 10C may be configured to obtain 14 the inhalation data from the server device 20, e.g., via a network, such as a global network as the internet, using the interface 10B. The inhalation data may be seen as an audio recording of an operation, such as an inhalation and/or an exhalation, performed by the user 1 with the inhaler device 30. In other words, the inhalation data may be seen as an audio recording of a sound sequence of an operation performed by the user 1 with the inhaler device 30, such as a sound output 32 produced by an inhalation, an exhalation, and/or an amplifier of the inhaler device 30.

The processor 10C is configured to determine, based on the inhalation data, using the predictor circuitry 12, a predicted inhalation parameter indicative of a prediction of an inhalation flow with the inhaler device 30.

In one or more example electronic devices and/or systems, the audio signal has a flow dependent sound frequency profile. In other words, the inhaler device 30 may be configured to generate a flow-dependent sound frequency profile that the audio signal is based on. For example, the inhaler device 30, such as acoustic inhaler device, may be configured to generate a sound output 32 (such as an audio signal) having a flow dependent sound frequency profile. The inhaler device 30 may comprise an acoustic amplifier, such as a whistle, configured to generate a sound output 32 (such as an audio signal) comprising a flow-dependent sound frequency profile. For example, the inhaler device 30 comprises a hole tone whistle configured to generate an audio signal comprising a flow-dependent sound frequency profile.

In one or more example electronic devices and/or systems, the electronic device 10 comprises one or more microphones 10D for obtaining (such as generating) the audio signal. For example, the electronic device 10 may comprise a mobile phone comprising one or more microphones 10D for obtaining the audio signal. In other words, the electronic device comprises one or more microphones 10D configured to generate and/or provide the audio signal based on a sound output 32 generated by the inhaler device 30, such as the acoustic inhaler device.

In one or more example electronic devices and/or systems, the obtaining of the inhalation data comprises to obtain, based on the audio signal, sound data of the audio signal, and wherein the determination of the predicted inhalation parameter is based on the sound data. In one or more example electronic devices and/or systems, the processor 10C is configured to obtain, based on the audio signal, sound data of the audio signal. In one or more example electronic devices and/or systems, the processor 10C is configured to determine the predicted inhalation parameter based on the sound data. In other words, the processor 10C may be configured to extract one or more audio features from the audio signal, where the sound data comprises the one or more audio features. It may be appreciated that the prediction model may be configured to extract one or more audio features from the audio signal to obtain (such as provide) the sound data. The sound data may comprise one or more audio features, such as a sound frequency feature and/or an amplitude feature.

In one or more example electronic devices and/or systems, the obtaining of the inhalation data comprises to apply one or more filters to the audio signal. In one or more example electronic devices and/or systems, the processor 10C may be configured to apply one or more filters to the audio signal to obtain, such as determine, the inhalation data. In other words, the processor 10C comprises a pre-processing module (not shown) configured to apply one or more filters to the audio signal. To apply one or more filters may comprise to apply a low-pass filter and/or a high-pass filter to the audio signal. By applying one or more filters to the audio signal the signal to noise ratio (SNR) may be enhanced. In one or more example electronic devices and/or systems, the prediction circuitry 12 is configured to apply the one or more filters by using the prediction model on the audio signal. In other words, the prediction model may be configured to (such as used to) extract the audio signal representing an inhalation and/or an exhalation with an inhaler device from an audio signal (such as audio data) by applying one or more filters.

In one or more example electronic devices and/or systems, the obtaining of the inhalation data comprises to identify a background noise (such as background sounds) from the audio signal. In one or more example electronic devices and/or systems, the processor 10C may be configured to identify a background noise from the audio signal. In one or more example electronic devices and/or systems, the prediction circuitry 12 is configured to identify a background noise by using the prediction model on the audio signal. For example, the prediction model may be pre-trained based on a plurality of background noises in order to be able to identify one or more background noises from the audio signal. In other words, the prediction model may be trained to extract the audio signal representing an inhalation and/or an exhalation with the inhaler device 30 from an audio signal (such as audio data) comprising one or more background noises (such as background sounds).

In one or more example electronic devices and/or systems, the obtaining of inhalation data comprises to split (such as cut) the audio signal (such as audio data) into a plurality of audio samples. In one or more example electronic devices and/or systems, the processor 10C is configured to split the audio signal into a plurality of audio samples. For example, the processor 10C may comprise an audio processing module (not shown) configured to process the audio signal, such as configured to split the audio signal into a plurality of audio samples. In other words, by splitting the audio signal (such as audio data) into a plurality of audio samples it may be possible to divide the audio signal into different phases of the inhalation and/or exhalation.

In one or more example electronic devices and/or systems, the determination of the predicted inhalation parameter is based on one or more audio samples of the plurality of audio samples. In one or more example electronic devices and/or systems, the processor 10C is configured to determine the predicted inhalation parameter based on one or more audio samples of the plurality of audio samples. In other words, the predictor circuitry 12 may be configured to provide one or more audio samples of the plurality of audio samples as an input to the prediction model. In other words, the processor 10C (such as the predictor circuitry 12) may be configured to provide the plurality of the audio samples as an input to the prediction model and keep the structure of the audio signal, such as keep the chronology of the audio samples.

In one or more example electronic devices and/or systems, the obtaining of inhalation data comprises to shuffle the plurality of audio samples. In one or more example electronic devices and/or systems, the processor 10C is configured to shuffle the plurality of audio samples. In one or more example electronic devices and/or systems, the determination of the predicted inhalation parameter is based on the shuffled audio samples.

In one or more example electronic devices and/or systems, the processor 10C is configured to determine the predicted inhalation parameter based on the shuffled audio samples. In other words, the predictor circuitry 12 may be configured to provide one or more of the shuffled audio samples of the plurality of audio samples as an input to the prediction model. In other words, the processor 10C (such as the predictor circuitry 12) may be configured to provide the plurality of shuffled audio samples as an input to the prediction model. By providing the plurality of shuffled audio samples as an input to the prediction model the prediction model may be trained to determine the predicted inhalation parameter without having the structure and/or chronology of the audio samples. This may provide a more robust prediction model to determine predicted inhalation parameters. Further, by splitting (such as cutting) and/or shuffling the audio signal, more audio data is provided, for example to train the prediction model, expand the audio data library, and/or determine predicted inhalation parameters on smaller audio samples. It may be appreciated that it may be possible to determine a plurality of predicted inhalation parameters based on the same inhalation (such as inhalation operation). It may be appreciated that the trained of the prediction model may be performed on the server device 20 or when the electronic device 10 acts as a server device on the electronic device 10.

In one or more example electronic devices and/or systems, the obtaining of inhalation data comprises to transform the audio signal to a spectrogram. In one or more example electronic devices and/or systems, the processor 10C is configured to transform the audio signal to a spectrogram.

In one or more example electronic devices and/or systems, the determination of the predicted inhalation parameter is based on the spectrogram. In one or more example electronic devices and/or systems, the processor 10C is configured to determine the predicted inhalation parameter based on the spectrogram.

In one or more example electronic devices and/or systems, the obtaining of the inhalation data comprises to obtain image data, such as based on the audio signal. In one or more example electronic devices and/or systems, the processor 10C is configured to obtain image data. In one or more example electronic devices and/or systems, the determination of the predicted inhalation parameter is based on the image data. In one or more example electronic devices and/or systems, the processor 10C is configured to determine the predicted inhalation parameter based on the image data. In other words, the processor 10C is configured to use the image data as input to the prediction model. In one or more example electronic devices and/or systems, the processor 10C is configured to determine one or more image features based on the image data. The one or more image features may be indicative of one or more of: an inhalation and/or exhalation phase (such as a phase of an operation of the user when taking medication with the inhaler device), an inhalation flow, an amplitude, time (such as duration), an inhalation volume, and an inhalation flow acceleration.

In one or more example electronic devices and/or systems, the processor 10C is configured to determine the predicted inhalation parameter based on the one or more features.

The processor 10C is configured to determine, based on the inhalation data, using the predictor circuitry 12, a predicted inhalation parameter indicative of a prediction of an inhalation flow with the inhaler device 30. An inhalation flow as disclosed herein may be seen as an inhalation flow rate. In other words, the processor 10C is configured to determine, based on the inhalation data, using the prediction model, a predicted inhalation parameter indicative of a prediction of an inhalation flow with the inhaler device 30. In one or more example electronic devices and/or systems, the processor 10C is configured to determine, based on the inhalation data, using the predictor circuitry 12, a predicted inhalation parameter indicative of an inhalation flow and/or an exhalation flow. Determining a predicted inhalation parameter indicative of a prediction of an inhalation flow may comprise extracting one or more inhalation and/or exhalation features from the audio signal and/or the inhalation data. In other words, determining a predicted inhalation parameter indicative of a prediction of an inhalation flow may be based on one or more features extracted from the audio signal and/or the inhalation data. In one or more example electronic devices and/or systems, the processor 10C is configured to determine the predicted inhalation parameter based on one or more features extracted from the audio signal and/or the inhalation data. For example, when the audio signal has a flow dependent sound frequency, the processor 10C may be configured to extract a sound frequency from the audio signal, and to determine an inhalation flow and/or an exhalation flow based on the extracted sound frequency.

In one or more example electronic devices and/or systems, the determination of the predicted inhalation parameter comprises to determine inhalation flow data. Inhalation flow data may for example comprise one or more of the features as described above, such as the one or more extracted features and/or the one or more inhalation and/or exhalation features. The determination of the inhalation flow data may comprise to determine a predicted inhalation flow, such as a predicted inhalation flow over time.

In one or more example electronic devices and/or systems, the processor 10C is configured to determine, based on the predicted inhalation parameter, one or more of: a duration of inhalation, an inhalation volume, an average inhalation flow, a maximum inhalation flow, a minimum inhalation flow, median inhalation flow, an inhalation flow acceleration, and one or more exhalation parameters.

The processor 10C is configured to determine, based on the predicted inhalation parameter, an inhalation representation. The inhalation representation may be seen as a representation indicative of all or part of the inhalation and/or exhalation performed by a user with the inhaler device. In other words, the inhalation representation may be seen as an inhaler device operation representation. The inhalation representation may be seen as and/or comprise an evaluation of an inhalation and/or an exhalation with the inhaler device 30, such as an inhalation evaluation. In other words, the inhalation representation may be seen as and/or comprise an evaluation of an operation of the user 1 when taking a medication with the inhaler device 30. In one or more example electronic devices and/or systems, the inhalation representation comprises an inhalation and/or exhalation score indicative of a performance of the user 1 when performing an inhalation and/or exhalation with the inhaler device 30, such as a performance of a medication intake. The inhalation representation may indicate whether an inhalation, an exhalation, and/or a medication intake was successful or not. For example, the score may be indicative of a successful inhalation, exhalation, and/or medication intake when the predicted inhalation parameter satisfies a criterion. For example, the processor 10C may be configured to determine whether the score satisfies a criterion (such as the first criterion). When the score is above or equal to a threshold (such as the first threshold), it may be determined that the score satisfies the criterion (such as first criterion).

In one or more example electronic devices and/or systems, the inhalation representation comprises a representation of the predicted inhalation parameter. For example, the inhalation representation may comprise a representation of an inhalation flow with the inhaler device, such as a graph or a plot of the inhalation flow over time, a spectrogram and/or the image data as described herein. The inhalation representation may comprise one or more of: a representation of a duration of inhalation, a representation of an inhalation volume, a representation of an inhalation flow profile, a representation of an average inhalation flow, a representation of a maximum inhalation flow, a representation of a minimum inhalation flow, a representation of a median inhalation flow, a representation of an inhalation flow acceleration, and a representation of one or more exhalation parameters. The inhalation representation may be indicative of the whole inhalation and/or exhalation with the inhaler device, and/or part of the inhalation and/or exhalation with the inhaler device 30, such as a phase of the inhalation and/or exhalation with the inhaler device 30.

In one or more example electronic devices and/or systems, the inhalation representation is indicative of an evaluation of the inhaler device 30. In other words, the processor 10C may be configured to determine a state of the inhaler device 30 based on the inhalation data and/or the audio signal.

An advantage of having an inhalation representation, e.g. after an inhalation, an exhalation, and/or an operation with the inhaler device 30 has been performed, may be that the user 1 of the electronic device 10 and/or the inhaler device 30 may see or be informed right after the inhalation, such as operation, about his/her performance or the outcome of the inhalation in relation to the predicted inhalation parameter. Therefore, when an inhalation was unsuccessful the user may be aware of it and may be able to repeat the inhalation and/or operation and/or improve his/her performance for the next inhalation and/or operation. Furthermore, the user 1 of the electronic device 10 and/or the inhaler device 30 may get a better feedback on his/her performance or on the outcome of the inhalation and/or operation. The inhalation representation may provide a gamification of the users' performances. The inhalation representation may for example increase the intelligibility of the feedback to the user 1, e.g. by being able to visualize an inhalation and/or operation with the inhaler device 30, and further to improve his/her inhalation technique by being able to visualize an improvement of performances.

Optionally, the processor 10C is configured to output, via the interface 10B, the inhalation representation. Optionally, the processor 10C is configured to output 13, via the interface 10B, the inhalation representation to the server device 20. Optionally, the processor 10C is configured to output 6, via the interface 10B, the inhalation representation to the user 1.

In one or more example electronic devices and/or systems, the processor 10C is configured to determine whether the predicted inhalation parameter satisfies a first criterion.

In one or more example electronic devices and/or systems, in accordance with the predicted inhalation parameter satisfying the first criterion, the processor 10C is configured to determine a first recommendation.

In one or more example electronic devices and/or systems, the processor 10C is configured to output, via the interface 10B, the first recommendation. Optionally, the processor 10C is configured to output 13, via the interface 10B, the first recommendation to the server device 20. Optionally, the processor 10C is configured to output 6, via the interface 10B, the first recommendation to the user 1.

In one or more example electronic devices and/or systems, the first criterion comprises a first threshold associated with a first inhalation flow and/or a first inhalation time.

Optionally, the electronic device 10 may be configured to output 6 (such as user output), such as via a display interface of the imaging device 10 and/or a separate display interface of a separate electronic device, a user interface comprising a plurality of user interface objects to a user 1 of the electronic device. The electronic device 10, such as the processor 10C, may be configured to output 6, such as via the interface 10B, the inhalation representation to the user 1. In other words, the electronic device 10, such as the processor 10C, may be configured to output 6, such as display, a user interface comprising the inhalation representation to the user 1.

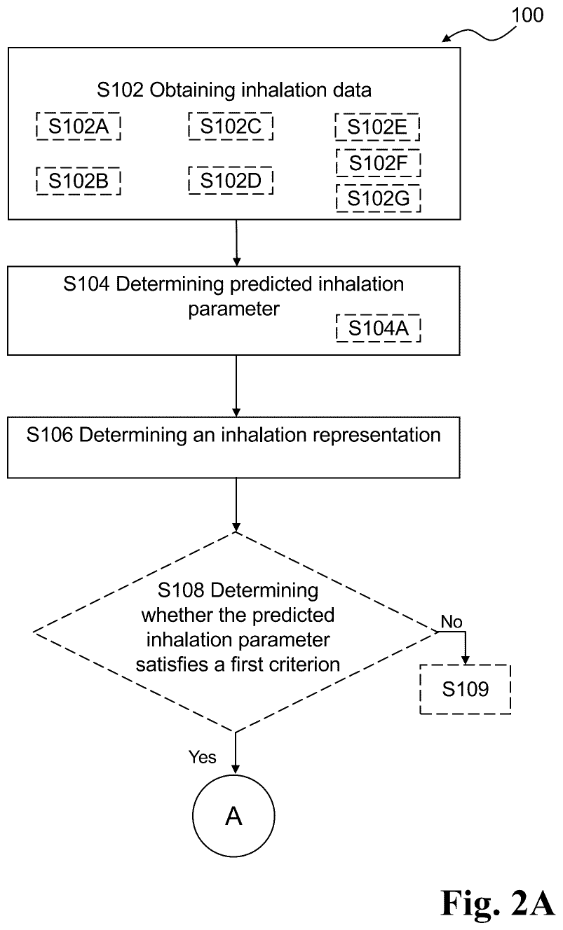

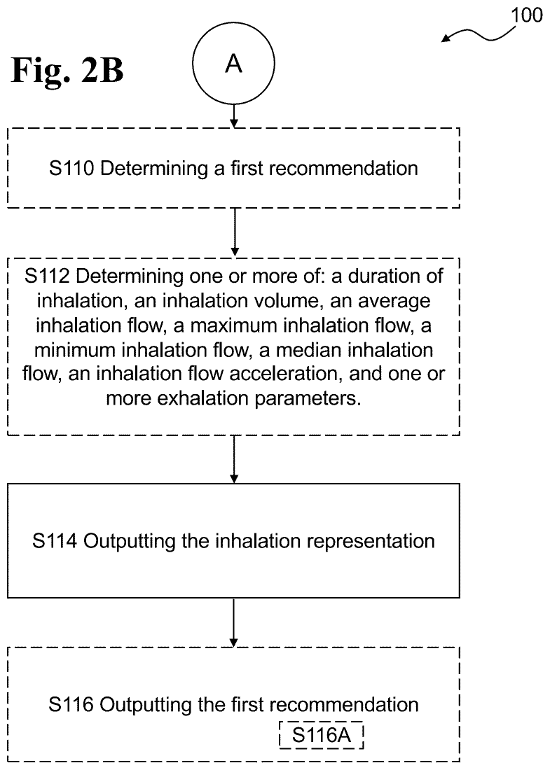

Optionally, the user 1 may provide an input 4 (such as user input), such as via the interface 10B, to the electronic device 10. The determination of one or more of the inhalation data, the predicted inhalation parameter, and the inhalation representation may be based on the input 4 from the user. The user 1 may for example provide an inhaler device type and/or user information about himself. The electronic device 10 may be configured to perform any of the methods disclosed in FIGS. 2A, 2B.

The processor 10C is optionally configured to perform any of the operations disclosed in FIGS. 2A-2B (such as any one or more of S102A, S102B, S102C, S102D, S102E, S102F, S102G, S104A, S108, S110, S112, S114, S116, S116A). The operations of the electronic device 10 may be embodied in the form of executable logic routines (for example, lines of code, software programs, etc.) that are stored on a non-transitory computer readable medium (for example, memory 10A) and are executed by the processor 10C).

Furthermore, the operations of the electronic device 10 may be considered a method that the electronic device 10 is configured to carry out. Also, while the described functions and operations may be implemented in software, such functionality may as well be carried out via dedicated hardware or firmware, or some combination of hardware, firmware and/or software.

Memory 10A may be one or more of a buffer, a flash memory, a hard drive, a removable media, a volatile memory, a non-volatile memory, a random access memory (RAM), or other suitable device. In a typical arrangement, memory 10A may include a non-volatile memory for long term data storage and a volatile memory that functions as system memory for the processor 10C. The memory 10A may exchange data with the processor 10C over a data bus. Control lines and an address bus between the memory 10B and the processor 10C also may be present (not shown in FIG. 1). The memory 10A is considered a non-transitory computer readable medium. The memory 10A may be configured to store information such as inhalation data, sound data, audio data, image data, predicted inhalation parameter(s), inhalation representation(s), recommendation(s), and/or prediction model(s) as disclosed herein in a part of the memory.

The server device 20, such as the processor 20C, may be configured to perform any of the operations performed by the electronic device 10, such as the processor 10C, as described herein. In other words, the description related to the processor 10C may apply to the description of the processor 20C. For example, when the electronic device 10 acts as a server device, the electronic device 10 and the server device 20 may be considered as one device.

FIGS. 2A and 2B show a flow diagram of an exemplary method, such as a method 100. A method 100, for characterizing and/or monitoring an inhalation and/or an exhalation with an inhaler device is disclosed. The method 100, may be performed by an electronic device and/or a system as disclosed herein (such as electronic device 10 and/or system 2). The method 100 comprises obtaining S102 inhalation data, where the inhalation data is indicative of an audio signal representing an inhalation and/or an exhalation with the inhaler device.

The method 100 comprises determining S104, based on the inhalation data, using the predictor circuitry, a predicted inhalation parameter indicative of a prediction of an inhalation flow with the inhaler device.

The method 100 comprises determining S106, based on the predicted inhalation parameter, an inhalation representation.

The method 100 comprises outputting S114, via the interface, the inhalation representation.

In one or more exemplary methods, the method 100 comprises determining S108 whether the predicted inhalation parameter satisfies a first criterion. In one or more exemplary methods, the method 100 comprises, in accordance with the predicted inhalation parameter satisfying the first criterion, determining S110, a first recommendation. In one or more exemplary methods, the method 100 comprises outputting S116, via the interface, the first recommendation.

In one or more exemplary methods, the method 100 comprises in accordance with the predicted inhalation parameter not satisfying the first criterion, determining S109, a second recommendation.

In one or more exemplary methods, the method 100 comprises outputting S116A, via the interface, the second recommendation.

In one or more exemplary methods, the obtaining S102 of the inhalation data comprises obtaining S102A, based on the audio signal, sound data of the audio signal. In one or more exemplary methods, the determination S104 of the predicted inhalation parameter is based on the sound data.

In one or more exemplary methods, the obtaining S102 of the inhalation data comprises applying S102B one or more filters to the audio signal.

In one or more exemplary methods, the obtaining S102 of the inhalation data comprises identifying S102C a background noise from the audio signal.

In one or more exemplary methods, the obtaining S102 of the inhalation data comprises splitting S102D the audio signal into a plurality of audio samples. In one or more exemplary methods, the determination S104 of the predicted inhalation parameter is based on one or more audio samples of the plurality of audio samples.

In one or more exemplary methods, the obtaining S102 of inhalation data comprises shuffling S102E the plurality of audio samples. In one or more exemplary methods the determination S104 of the predicted inhalation parameter is based on the shuffled audio samples.

In one or more exemplary methods, the obtaining S102 of inhalation data comprises transforming S102F the audio signal to a spectrogram. In one or more exemplary methods, the determination S104 of the predicted of the predicted inhalation parameter is based on the spectrogram.

In one or more exemplary methods, the obtaining S102 of inhalation data comprises obtaining S102G image data. In one or more exemplary methods, the determination S104 of the predicted inhalation parameter is based on the image data.

In one or more exemplary methods, the determination S106 of the predicted inhalation parameter comprises determining S104A inhalation flow data.

In one or more exemplary methods, the method 100 comprises determining S112, based on the predicted inhalation parameter, one or more of: a duration of inhalation, an inhalation volume, an average inhalation flow rate, a maximum inhalation flow rate, a minimum inhalation flow rate, median inhalation flow, an inhalation flow acceleration, and one or more exhalation parameters.

FIG. 3 shows an example representation of inhalation data indicative of an audio signal representing an inhalation with an inhaler device as disclosed herein. FIG. 3 is a graph representing frequency (such as sound frequency) in Hz on the Y-axis and inhalation flow (such as flow rate) in l/min on the X-axis. The amplitude in dB is represented with the color-coded scale on the right side of the graph (such as upper side). The inhalation data of FIG. 3 has been provided with a test setup where a pump has been connected to an inhaler device as disclosed herein and set to perform inhalations at different inhalation flows (such as constant inhalation flows). Each bar on the X-axis represents a different inhalation flow. The inhalation flows go from 6 l/min to 93 l/min. In other words, the graph of FIG. 3 represents snippets of inhalations performed with a test setup using a pump and an inhalation device as disclosed herein, the inhalations performed at inhalation flow rates between 6 l/min and 93 l/min. The inhalations at different inhalation flow rates are assembled in ascending order based on inhalation flow rate. FIG. 3 allows to visualize the relationship between inhalation flow rate, sound frequency profile, and sound amplitude for inhalations using an inhaler device as disclosed herein. Inhalation data as shown in FIG. 3 may be used to train the prediction model as disclosed herein.

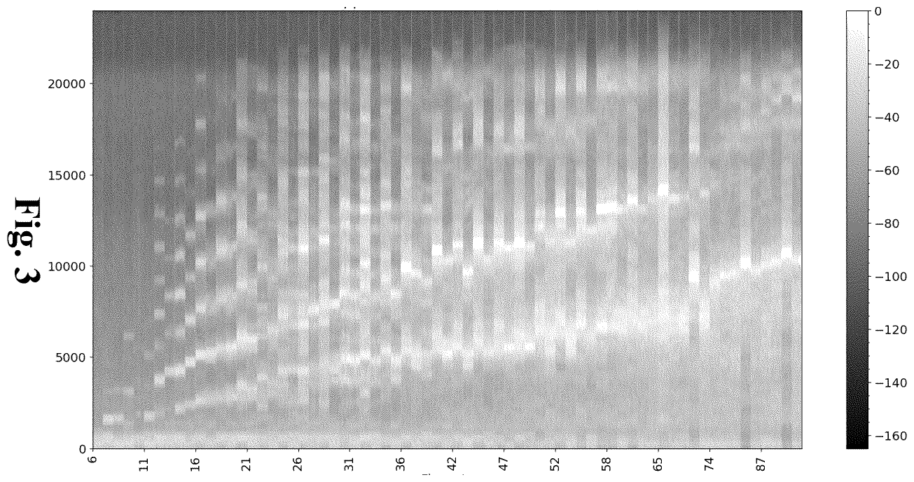

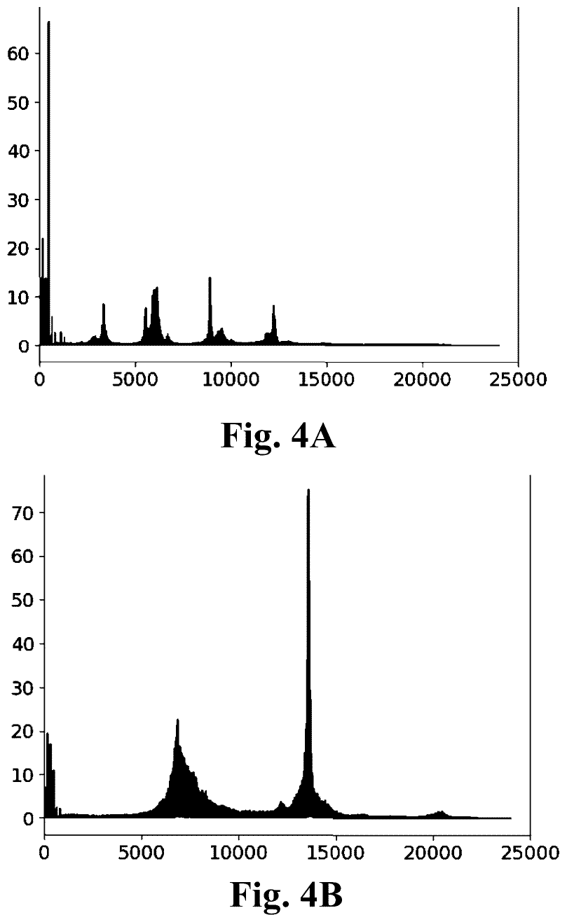

FIGS. 4A-4B show an example representation of inhalation data indicative of an audio signal representing an inhalation with an inhaler device as disclosed herein. FIGS. 4A-4B are two graphs representing frequency (such as sound frequency) in Hz on the X-axis and magnitude (such as energy) on the Y-axis. The magnitude is a relative difference in magnitude and is therefore unitless. The graph of FIG. 4A represents the magnitude with respect to frequency for an inhalation performed at an inhalation flow of 20 l/min. The graph of FIG. 4B represents the magnitude with respect to frequency for an inhalation performed at an inhalation flow of 60 l/min. As may be seen on FIGS. 4A-4B, different frequency peaks having different magnitudes are present for different inhalation flows.

The inhalation data of FIGS. 4A-4B has been provided with a test setup where a pump has been connected to an inhaler device as disclosed herein and set to perform inhalations at different inhalation flows (such as constant inhalation flows). Inhalation data as shown in FIGS. 4A-4B may be used to train the prediction model as disclosed herein, such as used as inputs to the prediction model.

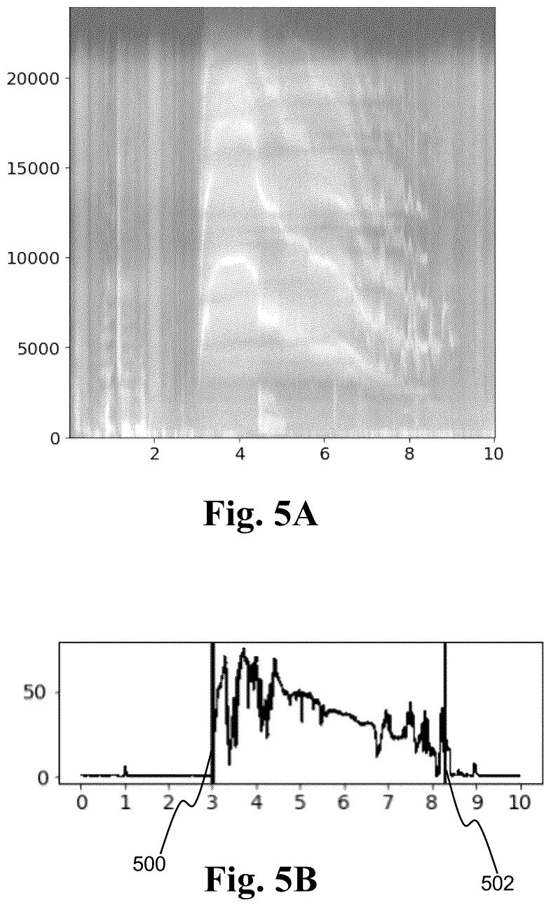

FIGS. 5A-5B show an example scenario of characterization and/or monitoring of an inhalation and/or an exhalation with an inhaler device where the technique disclosed herein is applied. In other words, FIGS. 5A-5B show an example scenario where an electronic device according to the present disclosure has been used to characterize and/or monitor an inhalation and/or an exhalation with an inhaler device. FIG. 5A shows a visualization (such as representation) of inhalation data indicative of an audio signal representing an inhalation and/or exhalation with the inhaler device. FIG. 5A is a graph representing an inhalation and/or exhalation with an inhaler device as disclosed herein where the frequency (such as sound frequency) in Hz is on the Y-axis and the time in seconds(s) is on the X-axis. The inhalation data is in FIG. 5A visualized (such as represented) in the form of a plot of an inhalation sound profile. The inhalation data has been obtained by the processor (such as processor 10C of FIG. 1). The processor of the electronic device has determined, based on the inhalation data represented in FIG. 5A, using the predictor circuitry, a predicted inhalation parameter indicative of a prediction of an inhalation flow with the inhaler device. The processor of the electronic device has determined, based on the predicted inhalation parameter, an inhalation representation. The inhalation representation is shown in FIG. 5B. The representation of FIG. 5B may be outputted via the interface of the electronic device, e.g., outputted to the user of the electronic device and/or inhaler device. FIG. 5B is a graph representing the determined inhalation representation. The graph of FIG. 5B represents on the Y-axis the predicted inhalation parameter indicative of a prediction of an inhalation flow with the inhaler device in l/min. The electronic device has determined, based on the predicted inhalation parameter, a duration of inhalation. As may be seen on FIG. 5B, the beginning 500 of the inhalation (such as an introductory inhalation phase) has been determined by the processor and the ending 502 of the inhalation (such as an ending phase) has been determined by the processor. The duration of the inhalation has been determined, based on the beginning 500 and ending 502 of the inhalation, to be 5.2 s. A duration of inhalation of at least 3 s may be seen as satisfying. Therefore a duration of inhalation of 5.2 s may be determined as being satisfying.

However, as may be seen in FIG. 5B, the inhalation flow is decreasing in time. The mean inhalation flow is above 40 l/min from 3 s to 4.5 s approximately and then the inhalation flow starts decreasing. Further, the inhalation flow comprises interruptions and fluctuations. The electronic device may therefore determine that the inhalation was not satisfying. A second recommendation may be determined by the electronic device based on the predicted inhalation parameter not satisfying a first criterion. The first criterion may for example comprise a first threshold associated with a first inhalation flow and/or first inhalation time. For example, to perform a successful inhalation, the inhalation flow may have to be above a first inhalation flow threshold for a certain period of time being above a first inhalation time (such as duration) threshold. The first threshold may comprise a first inhalation flow threshold of 20 l/min and/or a first inhalation time threshold of 3 s.

Since the inhalation flow in FIG. 5B has been predicted not to be above 20 l/min for at least 3 s, the inhalation illustrated in FIGS. 5A-5B is not satisfying. The inhalation is not continuous over time. The second recommendation may be determined to be “Your inhalation was not continuous over time, next time please repeat the inhalation operation where a constant inhalation flow is kept for at least 3 s”. The inhalation representation and/or the second recommendation may be outputted as feedback to the user of the inhaler device.

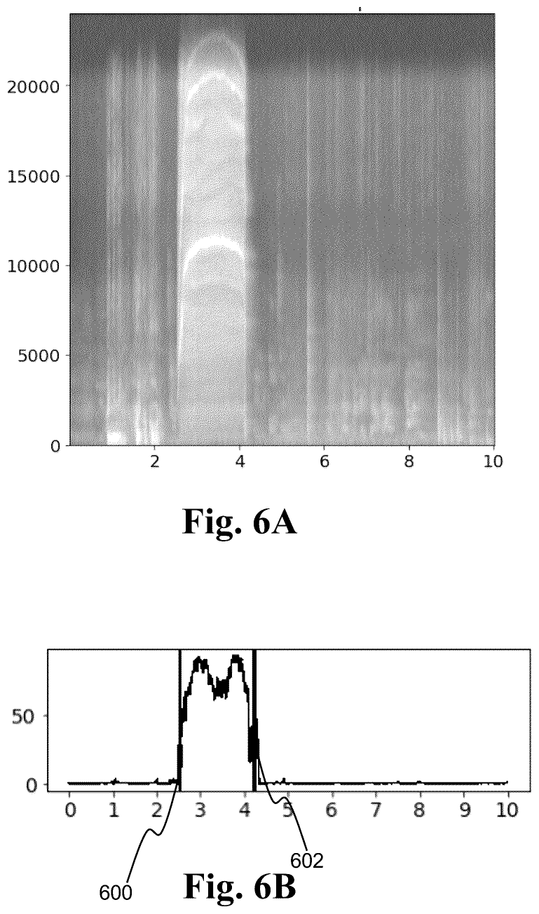

FIGS. 6A-6B show an example scenario of characterization and/or monitoring of an inhalation and/or an exhalation with an inhaler device where the technique disclosed herein is applied. In other words, FIGS. 6A-6B show an example scenario where an electronic device according to the present disclosure has been used to characterize and/or monitor an inhalation and/or an exhalation with an inhaler device. FIG. 6A shows a visualization (such as representation) of inhalation data indicative of an audio signal representing an inhalation and/or exhalation with the inhaler device. FIG. 6A is a graph representing an inhalation and/or exhalation with an inhaler device as disclosed herein where the frequency (such as sound frequency) in Hz is on the Y-axis and the time in seconds(s) is on the X-axis. The inhalation data is in FIG. 6A visualized (such as represented) in the form of a plot of an inhalation sound profile. The inhalation data has been obtained by the processor (such as processor 10C of FIG. 1). The processor of the electronic device has determined, based on the inhalation data represented in FIG. 6A, using the predictor circuitry, a predicted inhalation parameter indicative of a prediction of an inhalation flow with the inhaler device. The processor of the electronic device has determined, based on the predicted inhalation parameter, an inhalation representation. The inhalation representation is shown in FIG. 6B. The representation of FIG. 6B may be outputted via the interface of the electronic device, e.g., outputted to the user of the electronic device and/or inhaler device. FIG. 6B is a graph representing the determined inhalation representation. The graph of FIG. 6B represents on the Y-axis the predicted inhalation parameter indicative of a prediction of an inhalation flow with the inhaler device in l/min. The electronic device has determined, based on the predicted inhalation parameter, a duration of inhalation. As may be seen on FIG. 6B, the beginning 600 of the inhalation (such as an introductory inhalation phase) has been determined by the processor and the ending 602 of the inhalation (such as an ending phase) has been determined by the processor. As may be seen in FIG. 6B, the inhalation flow is rather constant in time during the inhalation. The mean inhalation flow is above 40 l/min during the inhalation. However, the duration of the inhalation has been determined, based on the beginning 600 and ending 602 of the inhalation, to be 1.6 s. A duration of inhalation of at least 3 s may be seen as satisfying. Therefore a duration of inhalation of 1.6 s may be determined as being unsatisfying. The electronic device may therefore determine that the inhalation was not satisfying. The second recommendation may be determined to be “Your inhalation was too short but the inhalation flow of your inhalation was satisfying, next time please repeat the inhalation operation where a constant inhalation flow is kept for at least 3 s. Please consider inhaling with less power but for a longer duration”. The inhalation representation and/or the second recommendation may be outputted as feedback to the user of the inhaler device.

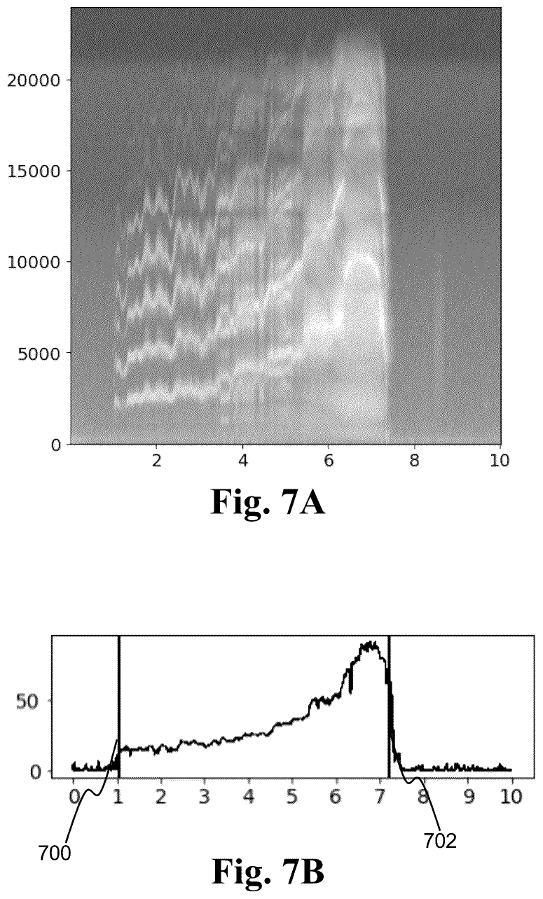

FIGS. 7A-7B show an example scenario of characterization and/or monitoring of an inhalation and/or an exhalation with an inhaler device where the technique disclosed herein is applied. In other words, FIGS. 7A-7B show an example scenario where an electronic device according to the present disclosure has been used to characterize and/or monitor an inhalation and/or an exhalation with an inhaler device. FIG. 7A shows a visualization (such as representation) of inhalation data indicative of an audio signal representing an inhalation and/or exhalation with the inhaler device. FIG. 7A is a graph representing an inhalation and/or exhalation with an inhaler device as disclosed herein where the frequency (such as sound frequency) in Hz is on the Y-axis and the time in seconds(s) is on the X-axis. The inhalation data is in FIG. 7A visualized (such as represented) in the form of a plot of an inhalation sound profile. The inhalation data has been obtained by the processor (such as processor 10C of FIG. 1). The processor of the electronic device has determined, based on the inhalation data represented in FIG. 7A, using the predictor circuitry, a predicted inhalation parameter indicative of a prediction of an inhalation flow with the inhaler device. The processor of the electronic device has determined, based on the predicted inhalation parameter, an inhalation representation. The inhalation representation is shown in FIG. 7B. The representation of FIG. 7B may be outputted via the interface of the electronic device, e.g., outputted to the user of the electronic device and/or inhaler device. FIG. 7B is a graph representing the determined inhalation representation. The graph of FIG. 7B represents on the Y-axis the predicted inhalation parameter indicative of a prediction of an inhalation flow with the inhaler device in l/min. The electronic device has determined, based on the predicted inhalation parameter, a duration of inhalation. As may be seen on FIG. 7B, the beginning 700 of the inhalation (such as an introductory inhalation phase) has been determined by the processor and the ending 702 of the inhalation (such as an ending phase) has been determined by the processor. The duration of the inhalation has been determined, based on the beginning 700 and ending 702 of the inhalation, to be 6.1 s. A duration of inhalation of at least 3 s may be seen as satisfying. Therefore a duration of inhalation of 6.1 s may be determined as being satisfying. However, as may be seen in FIG. 7B, the inhalation flow is fluctuating in time during the inhalation. The inhalation flow is starting out low and is increasing in time. The electronic device may therefore determine that the inhalation was not satisfying. The second recommendation may be determined to be “Your inhalation was not continuous over time, next time please repeat the inhalation operation where a constant inhalation flow is kept for at least 3 s. Please consider starting the inhalation with more powerful inhalation flow and then keep the inhalation flow constant for at least 3 s”. The inhalation representation and/or the second recommendation may be outputted as feedback to the user of the inhaler device.

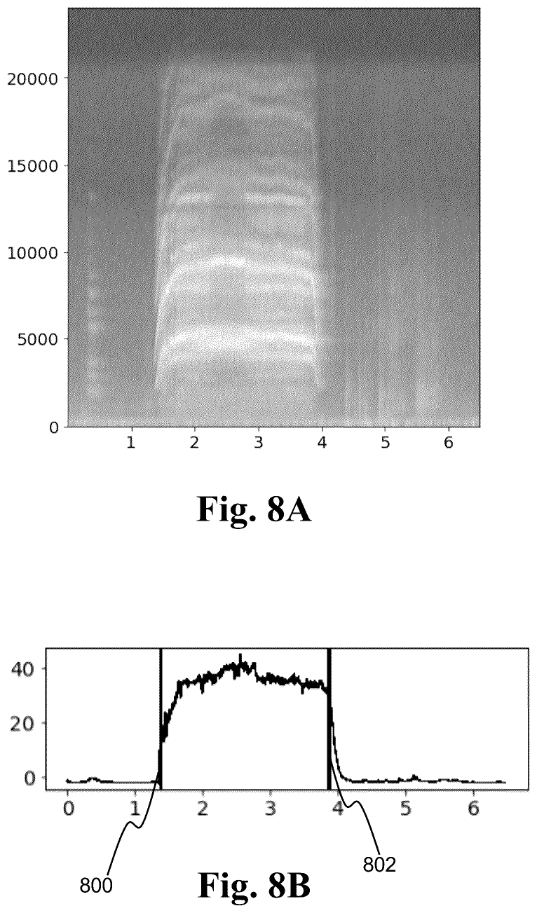

FIGS. 8A-8B show an example scenario of characterization and/or monitoring of an inhalation and/or an exhalation with an inhaler device where the technique disclosed herein is applied. In other words, FIGS. 8A-8B show an example scenario where an electronic device according to the present disclosure has been used to characterize and/or monitor an inhalation and/or an exhalation with an inhaler device. FIG. 8A shows a visualization (such as representation) of inhalation data indicative of an audio signal representing an inhalation and/or exhalation with the inhaler device. FIG. 8A is a graph representing an inhalation and/or exhalation with an inhaler device as disclosed herein where the frequency (such as sound frequency) in Hz is on the Y-axis and the time in seconds(s) is on the X-axis. The inhalation data is in FIG. 8A visualized (such as represented) in the form of a plot of an inhalation sound profile. The inhalation data has been obtained by the processor (such as processor 10C of FIG. 1). The processor of the electronic device has determined, based on the inhalation data represented in FIG. 8A, using the predictor circuitry, a predicted inhalation parameter indicative of a prediction of an inhalation flow with the inhaler device. The processor of the electronic device has determined, based on the predicted inhalation parameter, an inhalation representation. The inhalation representation is shown in FIG. 8B. The representation of FIG. 8B may be outputted via the interface of the electronic device, e.g., outputted to the user of the electronic device and/or inhaler device. FIG. 8B is a graph representing the determined inhalation representation. The graph of FIG. 8B represents on the Y-axis the predicted inhalation parameter indicative of a prediction of an inhalation flow with the inhaler device in l/min. The electronic device has determined, based on the predicted inhalation parameter, a duration of inhalation. As may be seen on FIG. 8B, the beginning 800 of the inhalation (such as an introductory inhalation phase) has been determined by the processor and the ending 802 of the inhalation (such as an ending phase) has been determined by the processor. The duration of the inhalation has been determined, based on the beginning 800 and ending 802 of the inhalation, to be 3 s. A duration of inhalation of at least 3 s may be seen as satisfying. Therefore a duration of inhalation of 3 s may be determined as being satisfying. Further, as may be seen in FIG. 8B, the inhalation flow is continuous in time during the inhalation. The inhalation flow is of at least 20 l/min for the entire inhalation duration. The electronic device may therefore determine that the inhalation was satisfying. The first recommendation may be determined to be “Your inhalation was satisfying. Continue like that”. The inhalation representation and/or the first recommendation may be outputted as feedback to the user of the inhaler device.

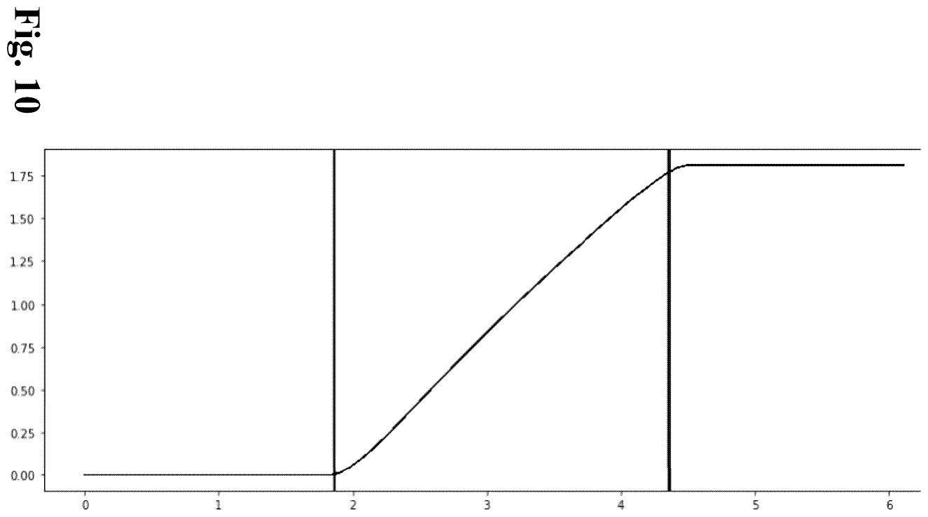

It may be appreciated that the electronic device may determine an inhalation volume by determining the area under the curve in the graphs of FIGS. 5B, 6B, 7B, 8B between the two vertical lines representing the beginning and the ending of the inhalation. This is illustrated in FIG. 10. It may be appreciated that an average absolute predicted inhalation parameter prediction error (in l/min) is 2.03 for an inhalation flow in the range of 20-60 l/min, 1.77 for an inhalation flow in the range of 20-90 l/min, and 2.29 for an inhalation flow in the range of 0-100 l/min.

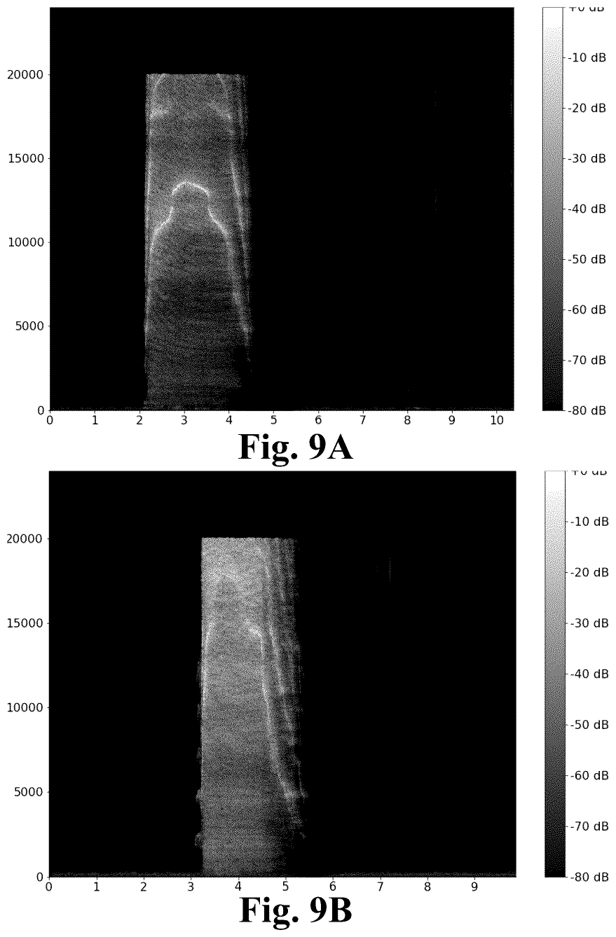

FIGS. 9A-9B show an example scenario of characterization and/or monitoring of an inhalation and/or an exhalation with an inhaler device where the technique disclosed herein is applied. In other words, FIGS. 9A-9B show an example scenario where an electronic device according to the present disclosure has been used to characterize and/or monitor an inhalation in FIG. 9A and an exhalation in FIG. 9B with an inhaler device. FIG. 9A shows a visualization (such as representation) of inhalation data indicative of an audio signal representing an inhalation with the inhaler device. FIG. 9A is a graph representing an inhalation with an inhaler device as disclosed herein where the frequency (such as sound frequency) in Hz is on the Y-axis and the time in seconds(s) is on the X-axis. The inhalation data is in FIG. 9A visualized (such as represented) in the form of a plot of an inhalation sound profile. The inhalation data has been obtained by the processor (such as processor 10C of FIG. 1). The processor of the electronic device has determined, based on the inhalation data represented in FIG. 9A, using the predictor circuitry, a predicted inhalation parameter indicative of a prediction of an inhalation flow with the inhaler device.

FIG. 9B shows a visualization (such as representation) of exhalation data indicative of an audio signal representing an exhalation with the inhaler device. FIG. 9B is a graph representing an exhalation with an inhaler device as disclosed herein where the frequency (such as sound frequency) in Hz is on the Y-axis and the time in seconds(s) is on the X-axis. The exhalation data is in FIG. 9B visualized (such as represented) in the form of a plot of an exhalation sound profile. The exhalation data has been obtained by the processor (such as processor 10C of FIG. 1). The processor of the electronic device has determined, based on the inhalation data represented in FIG. 9B, using the predictor circuitry, a predicted inhalation parameter indicative of a prediction of an exhalation flow with the inhaler device.

The processor of the electronic device has determined, based on the predicted inhalation parameter, that the audio data associated with FIG. 9A is indicative of an inhalation whereas the audio data associated with FIG. 9B is indicative of an exhalation.

FIG. 10 shows an example scenario of characterization and/or monitoring of an inhalation and/or an exhalation with an inhaler device where the technique disclosed herein is applied. The electronic device may be configured to determine, based on the predicted inhalation parameter, an inhalation volume. It may be appreciated that the electronic device may determine an inhalation volume by determining the area under the curve in the graphs of FIGS. 5B, 6B, 7B, 8B between the two vertical lines representing the beginning and the ending of the inhalation. For example, the inhalation volume may be calculated as the integral of predicted inhalation flow rate as a function of time. This is illustrated in FIG. 10.

In other words, FIG. 10 shows an example scenario where an electronic device according to the present disclosure has been used to characterize and/or monitor an inhalation with an inhaler device of metered dose inhaler type. FIG. 10 shows a visualization (such as representation) of an inhalation volume during an inhalation with the inhaler device. FIG. 10 is a graph representing the inhalation volume over time (such as cumulative inhalation volume as a function of time) during an inhalation with an inhaler device as disclosed herein where the inhalation volume in L (liters) is on the Y-axis and the time in seconds(s) is on the X-axis. The processor of the electronic device has determined, based on the inhalation data and based on the predicted inhalation parameter, an inhalation volume during an inhalation with the inhaler device. The electronic device may be configured to determine an inhalation representation based on the inhalation volume. The inhalation representation is illustrated in FIG. 10.

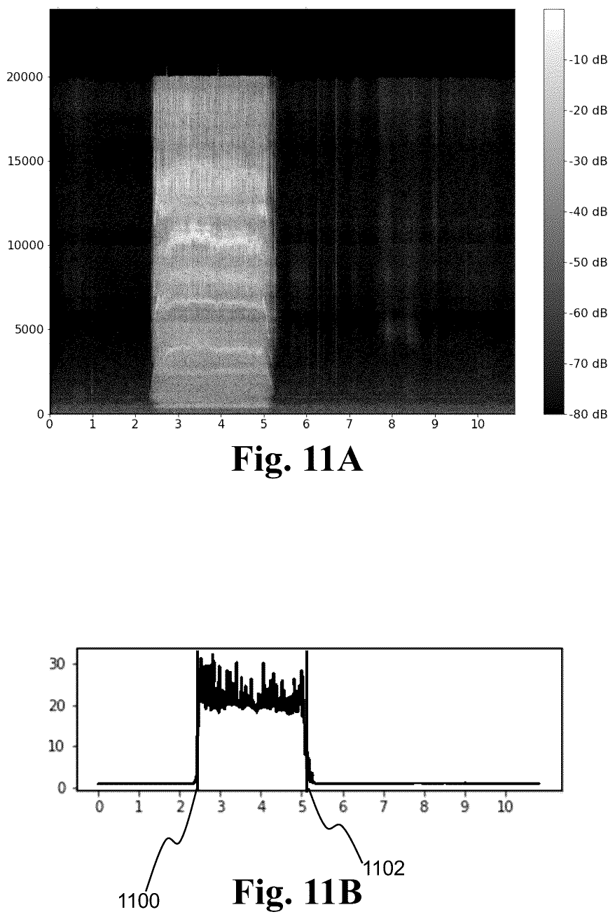

FIGS. 11A-11B show an example scenario of characterization and/or monitoring of an inhalation and/or an exhalation with an inhaler device where the technique disclosed herein is applied. In other words, FIGS. 11A-11B show an example scenario where an electronic device according to the present disclosure has been used to characterize and/or monitor an inhalation and/or an exhalation with an inhaler device of a capsule-based inhaler and with an acoustic amplifier. FIG. 11A shows a visualization (such as representation) of inhalation data indicative of an audio signal representing an inhalation and/or exhalation with the inhaler device. FIG. 11A is a graph representing an inhalation and/or exhalation with an inhaler device as disclosed herein where the frequency (such as sound frequency) in Hz is on the Y-axis and the time in seconds(s) is on the X-axis. The inhalation data is in FIG. 11A visualized (such as represented) in the form of a plot of an inhalation sound profile. The inhalation data has been obtained by the processor (such as processor 10C of FIG. 1). The processor of the electronic device has determined, based on the inhalation data represented in FIG. 11A, using the predictor circuitry, a predicted inhalation parameter indicative of a prediction of an inhalation flow with the inhaler device. The processor of the electronic device has determined, based on the predicted inhalation parameter, an inhalation representation. The inhalation representation is shown in FIG. 11B. The representation of FIG. 11B may be outputted via the interface of the electronic device, e.g., outputted to the user of the electronic device and/or inhaler device. FIG. 11B is a graph representing the determined inhalation representation. The graph of FIG. 11B represents on the Y-axis the predicted inhalation parameter indicative of a prediction of an inhalation flow with the inhaler device in l/min. The electronic device has determined, based on the predicted inhalation parameter, a duration of inhalation. As may be seen on FIG. 11B, the beginning 1100 of the inhalation (such as an introductory inhalation phase) has been determined by the processor and the ending 1102 of the inhalation (such as an ending phase) has been determined by the processor. The duration of the inhalation has been determined, based on the beginning 1100 and ending 1102 of the inhalation, to be 2.7 s. A duration of inhalation of at least 3 s may be seen as satisfying. Therefore a duration of inhalation of 2.7 s may be determined as being almost satisfying. Further, as may be seen in FIG. 11B, the inhalation flow is continuous in time during the inhalation. The inhalation flow is of at least 20 l/min for the entire inhalation duration. The electronic device may therefore determine that the inhalation was satisfying. The first recommendation may be determined to be “Your inhalation was satisfying. Continue like that”. The inhalation representation and/or the first recommendation may be outputted as feedback to the user of the inhaler device. As may be seen on FIGS. 11A-11B, the sound profile for a capsule-based inhaler with an acoustic amplifier is very different from the sound profiles of e.g., FIGS. 5A-5B, 6A-6B, 7B, 8A-8B that are based on inhalation data performed with a metered dose type inhaler. The inhalation flow prediction demonstrated that even at low inhalation flow rates the flow rate could be predicted.

Examples of electronic devices, systems, and methods according to the disclosure are set out in the following items:
Item 1. An electronic device for characterizing and/or monitoring an inhalation and/or an exhalation with an inhaler device, the electronic device comprising:
a memory;
an interface;
and a processor comprising predictor circuitry configured to operate according to a prediction model, wherein the processor is configured to:
   obtain inhalation data, where the inhalation data is indicative of an audio signal representing an inhalation and/or an exhalation with the inhaler device;
   determine, based on the inhalation data, using the predictor circuitry, a predicted inhalation parameter indicative of a prediction of an inhalation flow with the inhaler device;
   determine, based on the predicted inhalation parameter, an inhalation representation; and
   output, via the interface, the inhalation representation.
Item 2. Electronic device according to item 1, wherein the processor is configured to:
determine whether the predicted inhalation parameter satisfies a first criterion;
in accordance with the predicted inhalation parameter satisfying the first criterion, determine a first recommendation; and
output, via the interface, the first recommendation.
Item 3. Electronic device according to item 2, wherein the first criterion comprises a first threshold associated with a first inhalation flow and/or a first inhalation time.
Item 4. Electronic device according to any of items 2-3, wherein the first recommendation is comprised in the inhalation representation.
Item 5. Electronic device according to any of items 2-4, wherein the first recommendation comprises one or more of: an inhalation depth recommendation, an inhalation duration recommendation, an inhalation flow rate recommendation, and an inhalation preparation recommendation.
Item 6. Electronic device according to any of the previous items, wherein the audio signal has a flow dependent sound frequency profile.
Item 7. Electronic device according to any of the previous items, wherein the electronic device comprises one or more microphones for obtaining the audio signal.
Item 8. Electronic device according to any of the previous items, wherein the obtaining of the inhalation data comprises to obtain, based on the audio signal, sound data of the audio signal, and wherein the determination of the predicted inhalation parameter is based on the sound data.
Item 9. Electronic device according to any of the previous items, wherein the obtaining of the inhalation data comprises to apply one or more filters to the audio signal.
Item 10. Electronic device according to any of the previous items, wherein the obtaining of the inhalation data comprises to identify a background noise from the audio signal.
Item 11. Electronic device according to any of the previous items, wherein the predictor circuitry comprises a neural network module configured to operate according to a neural network.
Item 12. Electronic device according to item 11, wherein the neural network is a deep neural network, such as a convolutional neural network and/or a recurrent neural network.
Item 13. Electronic device according to any of the previous items, wherein the predictor circuitry comprises a regressor module configured to operate according to a regression model.
Item 14. Electronic device according to any of the previous items, wherein the obtaining of inhalation data comprises to split the audio signal into a plurality of audio samples and wherein the determination of the predicted inhalation parameter is based on one or more audio samples of the plurality of audio samples.
Item 15. Electronic device according to item 14, wherein the obtaining of inhalation data comprises to shuffle the plurality of audio samples and wherein the determination of the predicted inhalation parameter is based on the shuffled audio samples.
Item 16. Electronic device according to any of the previous items, wherein the obtaining of inhalation data comprises to transform the audio signal to a spectrogram, and wherein the determination of the predicted inhalation parameter is based on the spectrogram.
Item 17. Electronic device according to any of the previous items, wherein the obtaining of the inhalation data comprises to obtain image data, and wherein the determination of the predicted inhalation parameter is based on the image data.
Item 18. Electronic device according to any of the previous items, wherein the determination of the predicted inhalation parameter comprises to determine inhalation flow data.
Item 19. Electronic device according to any of the previous items, wherein the processor is configured to determine, based on the predicted inhalation parameter, one or more of: a duration of inhalation, an inhalation volume, an average inhalation flow rate, a maximum inhalation flow rate, a minimum inhalation flow rate, median inhalation flow, an inhalation flow acceleration, and one or more exhalation parameters.
Item 20. Electronic device according to any of the previous items, wherein the electronic device is a user equipment device.
Item 21. Electronic device according to any of the previous items, wherein the electronic device is a server device.
Item 22. A system for characterizing and/or monitoring an inhalation and/or an exhalation with an inhaler device, the system comprising the inhaler device and an electronic device according to any of items 1-21.
Item 23. A method for characterizing and/or monitoring an inhalation and/or an exhalation with an inhaler device, the method (100) comprising:
obtaining (S102) inhalation data, where the inhalation data is indicative of an audio signal representing an inhalation and/or an exhalation with the inhaler device,
determining (S104), based on the inhalation data, using the predictor circuitry, a predicted inhalation parameter indicative of a prediction of an inhalation flow with the inhaler device,
determining (S106), based on the predicted inhalation parameter, an inhalation-representation; and
outputting (S114), via the interface, the inhalation representation.
Item 24. The method according to item 23, wherein the method comprises:
determining (S108) whether the predicted inhalation parameter satisfies a first criterion,
in accordance with the predicted inhalation parameter satisfying the first criterion, determining (S110) a first recommendation, and
outputting (S116), via the interface, the first recommendation.
Item 25. The method according to any of items 23-24, wherein the obtaining (S102) of the inhalation data comprises obtaining (S102A), based on the audio signal, sound data of the audio signal, and where the determination (S104) of the predicted inhalation parameter is based on the sound data.
Item 26. The methods according to any of items 23-25, wherein the obtaining (S102) of the inhalation data comprises applying (S102B) one or more filters to the audio signal.
Item 27. The methods according to any of items 23-26, wherein the obtaining (S102) of the inhalation data comprises removing (S102C) a background noise from the audio signal.
Item 28. The methods according to any of items 23-27, wherein the obtaining (S102) of the inhalation data comprises splitting (S102D) the audio signal into a plurality of audio samples, and where the determination (S104) of the predicted inhalation parameter is based on one or more audio samples of the plurality of audio samples.
Item 29. The methods according to item 28, wherein the obtaining (S102) of the inhalation data comprises shuffling (S102E) the plurality of audio samples, and where the determination (S104) of the predicted inhalation parameter is based on the shuffled audio samples.
Item 30. The methods according to any of items 23-29, wherein the obtaining (S102) of the inhalation data comprises transforming (S102F) the audio signal to a spectrogram, and where the determination (S104) of the predicted of the predicted inhalation parameter is based on the spectrogram.
Item 31. The methods according to any of items 23-30, wherein the obtaining (S102) of the inhalation data comprises obtaining (S102G) image data, and where the determination (S104) of the predicted inhalation parameter is based on the image data.
Item 32. The methods according to any of items 23-31, wherein the determination (S106) of the predicted inhalation parameter comprises determining (S104A) inhalation flow data.
Item 33. The methods according to any of items 23-32, wherein the method comprises: determining (S112), based on the predicted inhalation parameter, one or more of: a duration of inhalation, an inhalation volume, an average inhalation flow rate, a maximum inhalation flow rate, a minimum inhalation flow rate, median inhalation flow, an inhalation flow acceleration, and one or more exhalation parameters.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering. Furthermore, the labelling of a first element does not imply the presence of a second element and vice versa.

It may be appreciated that the Figures comprise some circuitries or operations which are illustrated with a solid line and some circuitries, components, features, or operations which are illustrated with a dashed line. Circuitries or operations which are comprised in a solid line are circuitries, components, features or operations which are comprised in the broadest example. Circuitries, components, features, or operations which are comprised in a dashed line are examples which may be comprised in, or a part of, or are further circuitries, components, features, or operations which may be taken in addition to circuitries, components, features, or operations of the solid line examples. It should be appreciated that these operations need not be performed in order presented. Furthermore, it should be appreciated that not all of the operations need to be performed. The example operations may be performed in any order and in any combination. It should be appreciated that these operations need not be performed in order presented. Circuitries, components, features, or operations which are comprised in a dashed line may be considered optional.

Other operations that are not described herein can be incorporated in the example operations. For example, one or more additional operations can be performed before, after, simultaneously, or between any of the described operations.

Certain features discussed above as separate implementations can also be implemented in combination as a single implementation. Conversely, features described as a single implementation can also be implemented in multiple implementations separately or in any suitable sub-combination. Moreover, although features may be described above as acting in certain combinations, one or more features from a claimed combination can, in some cases, be excised from the combination, and the combination may be claimed as any sub-combination or variation of any sub-combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the examples may be implemented at least in part by means of both hardware and software, and that several “means”, “units” or “devices” may be represented by the same item of hardware.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly, to be regarded in an illustrative rather than restrictive sense. The claimed disclosure is intended to cover all alternatives, modifications, and equivalents.

### LIST OF REFERENCES

1 user
2 system
4 input
6 inhalation representation, output
10 electronic device
10A one or more interfaces
10B memory
10C processor
10D one or more microphones
12 predictor circuitry
12A neural network module
12B regressor module
13 control, transmit
14 obtain
20 server device
20A memory
20B one or more interfaces
20C processor(s)
30 inhaler device
32 sound output
500, 600, 700, 800 inhalation beginning
502, 602, 702, 802 inhalation ending
100 method for characterizing and/or monitoring an inhalation and/or an exhalation with an inhaler device
S102 obtaining inhalation data
S102A obtaining, based on the audio signal, sound data of the audio signal
S102B applying one or more filters to the audio signal
S102C identifying a background noise from the audio signal
S102D splitting the audio signal into a plurality of audio samples
S102E shuffling the plurality of audio samples
S102F transforming the audio signal to a spectrogram
S102G obtaining image data
S104 determining, based on the inhalation data, using the predictor circuitry, a predicted inhalation parameter indicative of a prediction of an inhalation flow with the inhaler device
S104A determining inhalation flow data
S106 determining, based on the predicted inhalation parameter, an inhalation representation
S108 determining whether the predicted inhalation parameter satisfies a first criterion
S109 determining a second recommendation
S110 determining a first recommendation
S112 determining, based on the predicted inhalation parameter, one or more of: a duration of inhalation, an inhalation volume, an average inhalation flow rate, a maximum inhalation flow rate, a minimum inhalation flow rate, median inhalation flow, an inhalation flow acceleration, and one or more exhalation parameters
S114 outputting the inhalation representation
S116 outputting the first recommendation
S116A outputting the second recommendation

1. An electronic device for at least one of characterizing or monitoring at least one of an inhalation or an exhalation with an inhaler device, the electronic device comprising:
a memory;
an interface; and
a processor including predictor circuitry configured to operate according to a prediction model, wherein the processor is configured to
obtain inhalation data, wherein the inhalation data is indicative of an audio signal representing at least one of an inhalation or an exhalation with the inhaler device,
determine, based on the inhalation data, using the predictor circuitry, a predicted inhalation parameter indicative of a prediction of an inhalation flow with the inhaler device,
determine, based on the predicted inhalation parameter, an inhalation representation, and
output, via the interface, the inhalation representation.

2. The electronic device according to claim 1, wherein the processor is configured to:
determine whether the predicted inhalation parameter satisfies a first criterion;
in accordance with the predicted inhalation parameter satisfying the first criterion, determine a first recommendation; and
output, via the interface, the first recommendation.

3. The electronic device according to claim 2, wherein the first criterion comprises a first threshold associated with at least one of a first inhalation flow or a first inhalation time.

4. The electronic device according to claim 2, wherein the first recommendation is comprised in the inhalation representation.

5. The electronic device according to claim 2, wherein the first recommendation comprises one or more of: an inhalation depth recommendation, an inhalation duration recommendation, an inhalation flow recommendation, or an inhalation preparation recommendation.

6. The electronic device according to claim 1, wherein the audio signal has a flow dependent sound frequency profile.

7. The electronic device according to claim 1, wherein the electronic device comprises one or more microphones for obtaining the audio signal.

8. The electronic device according to claim 1, wherein the obtaining of the inhalation data comprises to obtain, based on the audio signal, sound data of the audio signal, and wherein the determination of the predicted inhalation parameter is based on the sound data.

9. The electronic device according to claim 1, wherein the obtaining of the inhalation data comprises to apply one or more filters to the audio signal.

10. The electronic device according to claim 1, wherein the obtaining of the inhalation data comprises to identify a background noise from the audio signal.

11. The electronic device according to claim 1, wherein the predictor circuitry comprises a neural network module configured to operate according to a neural network.

12. The electronic device according to claim 11, wherein the neural network is a deep neural network, which includes at least one of a convolutional neural network or a recurrent neural network.

13. The electronic device according to claim 1, wherein the predictor circuitry comprises a regressor module configured to operate according to a regression model.

14. The electronic device according to claim 1, wherein the obtaining of inhalation data comprises to split the audio signal into a plurality of audio samples and wherein the determination of the predicted inhalation parameter is based on one or more audio samples of the plurality of audio samples.

15. The electronic device according to claim 14, wherein the obtaining of inhalation data comprises to shuffle the plurality of audio samples and wherein the determination of the predicted inhalation parameter is based on the shuffled audio samples.

16. The electronic device according to claim 1, wherein the obtaining of inhalation data comprises to transform the audio signal to a spectrogram, and wherein the determination of the predicted inhalation parameter is based on the spectrogram.

17. The electronic device according to claim 1, wherein the obtaining of the inhalation data comprises to obtain image data, and wherein the determination of the predicted inhalation parameter is based on the image data.

18. The electronic device according to claim 1, wherein the determination of the predicted inhalation parameter comprises to determine inhalation flow data.

19. The electronic device according to claim 1, wherein the processor is configured to determine, based on the predicted inhalation parameter, one or more of: a duration of inhalation, an inhalation volume, an average inhalation flow, a maximum inhalation flow, a minimum inhalation flow, a median inhalation flow, an inhalation flow acceleration, or one or more exhalation parameters.

20.-22. (canceled)

23. A method for at least one of characterizing or monitoring at least one of an inhalation or an exhalation with an inhaler device, the method comprising:
obtaining inhalation data, wherein the inhalation data is indicative of an audio signal representing at least one of an inhalation or an exhalation with the inhaler device,
determining, based on the inhalation data, using predictor circuitry, a predicted inhalation parameter indicative of a prediction of an inhalation flow with the inhaler device,
determining, based on the predicted inhalation parameter, an inhalation representation; and
outputting, via an interface, the inhalation representation.

24.-33. (canceled)
